(19) United States
(12) Patent Application Publication
(10) Pub. No.: US 2005/0176813 A1
(43) Pub. Date: Aug. 11, 2005

(54) Novel chalcone derivatives and uses thereof

(76) Inventors: Jonathan B. Baell, Victoria (AU); Heike Wulff, Davis, CA (US); George K. Chandy, Irvine, CA (US); Raymond S. Norton, Victoria (AU)

Correspondence Address: SCULLY SCOTT MURPHY & PRESSER, PC, 400 GARDEN CITY PLAZA, SUITE 300, GARDEN CITY, NY, 11530, US

(21) Appl. No.: 10/507,782

(22) PCT Filed: Mar. 14, 2003
(86) PCT No.: PCT/AU03/00308

(30) Foreign Application Priority Data
Mar. 14, 2002 (AU) PS 1103

(51) Int. Cl. A61K 31/343

(52) U.S. Cl. 514/469

(57) ABSTRACT

Various chalcone derivatives of the general formula (I) are described and the variables, A, B, m and R¹ to R¹⁰ are as defined in the specification. These derivatives can be useful in the modulation of potassium channel activity in cells and may be useful in the treatment or prevention of autoimmune and inflammatory diseases.

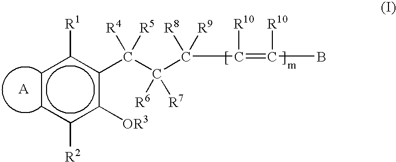

### FIELD OF THE INVENTION

The present invention relates to compounds useful in the modulation of potassium channel activity in cells, in particular the activity of Kv1.3 channels found in T cells. The invention also relates to the use of these compounds in the treatment or prevention of autoimmune and inflammatory diseases, including multiple sclerosis, pharmaceutical compositions containing these compounds and methods for their preparation.

### BACKGROUND

Many autoimmune and chronic inflammatory diseases are related to immunoregulatory abnormalities. Diseases such as systemic lupus erythematosis, chronic rheumatoid arthritis, multiple sclerosis and psoriasis have in common the appearance of autoantibodies and self-reactive lymphocytes.

Multiple sclerosis is the most common neurological disease of young people. It is believed to cost more in medical care and lost income than any other neurological disease of young adults.

Multiple sclerosis affects the myelin sheaths of nerves. Myelin is an insulating material that coats most axons and allows rapid signal conduction over long distances by saltatory conduction. It is thought that antibodies and specialised cells of the immune system attack the myelin coating. This process leads to inflammation and scarring (sclerosis) which damages blood vessels in the area by the formation of a lesion known as a plaque. These plaques are characterised by being infiltrated by cells of the immune system. This results in demyelination with the consequential loss of the rapid signal conduction.

Rheumatoid arthritis involves an inflammation in the lining of the joints and/or other internal organs. It is a systemic disease that affects the entire body, and as such it will typically affect many different joints. It is one of the most common forms of arthritis, and is characterized by the inflammation of the membrane lining the joint, which causes pain, stiffness, warmth, redness and swelling. The inflamed joint lining, known as the synovium, can invade and damage bone and cartilage. The inflammation can cause the release of enzymes that may attack bone and cartilage. The involved joint can lose its shape and alignment, resulting in pain and loss of movement.

A possible method of treating these autoimmune and inflammatory diseases is by suppressing T cell proliferation and modulating their activation.

The early stages of T-cell activation may be conceptually separated into pre-Ca²⁺ and post-Ca²⁺ events (Cahalan and Chandy 1997, Curr. Opin. Biotechnol. 8: 749). Following engagement of antigen with the T-cell antigen-receptor, activation of tyrosine kinases and the generation of inositol 1,4,5-triphosphate lead to the influx of Ca²⁺ and the rise of cytoplasmic Ca 2+concentration. The rise in Ca²⁺ activates the phosphatase calcineurin, which then dephosphorylates a cytoplasmically localized transcription factor (N-FAT) enabling it to accumulate in the nucleus and bind to a promoter element of the interleukin-2 gene. Along with parallel events involving the activation of protein kinase C and ras, gene transcription leads to lymphokine secretion and to lymphocyte proliferation. Some genes require long-lasting Ca²⁺ signals while others require only a transient rise of Ca²⁺. Furthermore, Ca²⁺ immobilisation of the T-cell at the site of antigen presentation helps to cement the interaction between T-cell and the antigen-presenting cell and thereby facilitate local signalling between the cells.

Ion channels underlie the Ca²⁺ signal of T-lymphocytes. Ca²⁺ ions move across the plasma membrane through a channel termed the store-operated Ca²⁺ channel or the calcium release-activated Ca²⁺ channel. Two distinct types of potassium channels indirectly determine the driving force of calcium entry. The first is the voltage-gated Kv1.3 channel (Cahalan 1985, J. Physiol. 385: 197; Grissmer 1990, Proc. Natl. Acad. Sci. USA 87: 9411; Verheugen 1995, J. Gen. Physiol. 105: 765; Aiyar 1996, J. Biol. Chem. 271: 31013; Cahalan and Chandy 1997, Curr. Opin. Biotechnol. 8: 749) and the second is the intermediate-conductance calcium-activated potassium channel, IKCa1 (Grissmer 1993, J. Gen. Physiol. 102: 601; Fanger 1999 J. Biol. Chem. 274: 5746; Rauer 1999, J. Biol. Chem. 274: 21885; VanDorpe 1998, J. Biol. Chem. 273: 21542; Joiner 1997, Proc. Natl. Acad. Sci. USA 94: 11013; Khanna 1999, J. Biol. Chem. 274: 14838; Lodgson 1997, J. Biol. Chem. 272: 32723; Ghanshani 1998, Genomics 51: 160). When these potassium channels open, the resulting efflux of K⁺ hyperpolarizes the membrane, which in turn accentuates the entry of Ca²⁺, which is absolutely required for downstream activation events (Cahalan and Chandy 1997, Cur. Opin. Biotechnol. 8: 749).

The predominant voltage-gated channel in human T-lymphocytes is encoded by Kv1.3, a Shaker-related gene. Kv1.3 has been characterised extensively at the molecular and physiological level and plays a vital role in controlling T-lymphocyte proliferation, mainly by maintaining the resting membrane potential of resting T-lymphocytes. Inhibition of this channel depolarises the cell membrane sufficiently to decrease the influx of Ca²⁺ and thereby prevents downstream activation events. The Kv1.3 channel is a homotetramer, consisting of 4 identical Kv1.3 subunits which are assembled to form the functional channel. Advantageously, the homotetrameric Kv1.3 channel is almost exclusively located in T-lymphocytes.

Compounds which are selective Kv1.3 blockers are thus potential therapeutic agents as immunosuppressants for the prevention of graft rejection, and the treatment of autoimmune and inflammatory disorders. They could be used alone or in conjunction with other immunosuppressants, such as selective IKCa1 blockers or cyclosporin, in order to achieve synergism and/or to reduce toxicity, especially of cyclosporin.

At present there exist a number of non-selective K channels that will inhibit lymphocyte proliferation, but have adverse side effects. Other K channels exist in a wide range of tissues including the heart and brain, and generally blocking these channels is undesirable.

U.S. Pat. No. 5,494,895 discloses the use of a thirty-nine amino acid peptide, scorpion peptide margatoxin, as a selective inhibitor and probe of Kv1.3 channels present in human lymphocytes, and also as an immunosuppressant. However the use of this compound is limited by its potent toxicity.

International Patent Application publication Nos. WO 97/16438 and WO 09/716,437, and U.S. Pat. No. 6,051,590 describe the use of the triterpene, correolide and related compounds as immunosuppressants in the treatment of conditions in mammals affected or facilitated by Kv1.3 inhibition.

U.S. Pat. No. 6,077,680 describes DNA segments and proteins of derived from sea anemone species, more particularly ShK toxin from Stichodactyla helianthus. The ShK toxin was found to block Kv1.1, Kv1.3, Kv1.4 and Kv1.6, but a mutant ShK-K22DAP found to selectively block Kv1.3. Unfortunately the mutant was not sufficiently stable for clinic use.

ShK toxin has been shown to both prevent and treat experimental autoimmune encephalomyelitis in Lewis rats, an animal model for human multiple sclerosis (Beeton 2001, et al., Proc. Natl. Acad. Sci. USA 98:13942), by selectively targeting T-cells chronically activated by the myelin antigen, MBP (myelin basic protein). The same study also indicated that chronically activated encephalitogenic rat T cells express a unique channel phenotype characterised by high expression of Kv1.3 channels (approximately 1500 per cell) and low numbers of IKCa1 channels (approximately 120 per cell). This channel phenotype is distinct from that seen in quiescent and acutely activated cells and may be a functionally relevant marker for chronically activated rat T-lymphocytes.

Recently khellinone, a substituted benzofuran and natural product from certain plants, and 8-Methoxypsoralen (8-MOP), both commercially available products, were found to have blocking activity on the Kv1.3 channel.

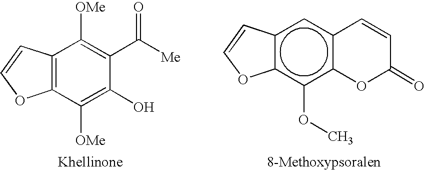

WO 01/726680 (Cancer Research Ventures Limited) describes a number of substituted chalcones, of the general formula 1-(4-methoxyphenyl)-3-(3,5-dimethoxyphenyl)prop-1-en-3-ones 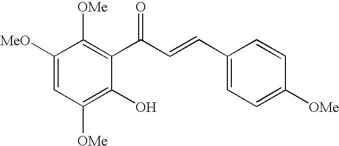
for use in the treatment of antiproliferative conditions such as cancer, and anti-inflammatory conditions such as rheumatoid arthritis. Chalcone is 1,3-diphenyl-2-propen-1-one.

### SUMMARY OF THE INVENTION

The invention relates to compounds of the general formula I 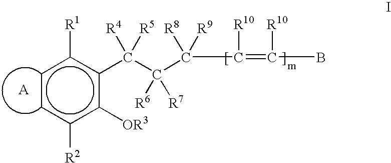
Where:—
ring A is an optionally substituted fused carbocyclic or heterocyclic ring;
B is an optionally substituted aromatic or heteroaromatic ring;
R¹ and R² are independently selected from hydrogen, cyano, halo, nitro, optionally substituted alkyl, optionally substituted alkenyl, optionally substituted alkynyl, optionally substituted cycloalkyl, —OR, —C(O)R, —C(O)OR, —OC(O)R (where R is hydrogen or selected from an optionally substituted alkyl, alkenyl, alkynyl, cycloalkyl and aryl group), —C(O)NR′R″, —NR′C(O)R″ and —NR′R″ (where R′ and R″ are independently selected from hydrogen or lower alkyl);
R³ is hydrogen or optionally substituted alkyl, alkenyl or alkynyl group;
R⁴ and R⁵ are independently selected from hydrogen, hydroxy, alkyl, alkenyl; alkynyl and alkoxy;
or R⁴ and R⁵ together are ═O, ═S, ═NR or ═NOR, (where R is hydrogen or lower alkyl);
R⁶ and R⁷ are independently selected from hydrogen, cyano, halo, nitro, optionally substituted alkyl, optionally substituted alkenyl, optionally substituted alkynyl optionally substituted cycloalkyl, —OR, —C(O)R, —C(O)OR, —OC(O)R (where R is from hydrogen or is selected from an optionally substituted alkyl, alkenyl, alkynyl, cycloalkyl and aryl group), —C(O)NR′R″ and —NR′R″ (where R′ and R″ are independently selected from hydrogen and lower alkyl);
or R³ together with R⁷ together with the atoms to which they are attached form an optionally substituted five or six membered heterocyclic ring;
R⁸ and R⁹ are independently selected from hydrogen, cyano, halo, nitro, a 5- or 6-membered nitrogen containing heterocyclic ring, optionally substituted alkyl, optionally substituted alkenyl, optionally substituted alkynyl, optionally substituted cycloalkyl, optionally substituted arylalkyl, optionally substituted heterocyclylalkyl, —OR, —C(O)R, —C(O)OR, —OC(O)R (where R is hydrogen or is selected from an optionally substituted alkyl, alkenyl, alkynyl, cycloalkyl and aryl group), —C(O)NR′R″, —NR′C(O)R″ and —NR′R″ (where R′ and R″ are independently selected from hydrogen and lower alkyl);
or R⁸ and R⁹ are together ═O, ═S, ═NR or ═NOR, (where R is hydrogen or lower alkyl);
or R⁶ and R⁸ together form a bond;
or R⁴, R⁵, R⁶, R⁸ and R⁹ together with the atoms to which they are attached form an aromatic or heteroaromatic ring;
or R⁶, R⁷ and R⁸ and the atoms to which they are attached, together with a ring atom of B form a six membered aromatic or heteroaromatic ring fused to ring B;
m=0, 1 or 2;
each R¹⁰ is independently selected from hydrogen, cyano, halo, nitro, optionally substituted alkyl, optionally substituted alkenyl, optionally substituted alkynyl and optionally substituted cycloalkyl;
with the proviso that R³ is not —CH₂CO₂H when R¹ and R² are methoxy, m is 0, R⁴ and R⁵ together are ═O, R⁶ and R⁸ together form a bond, R⁷ and R⁹ are hydrogen, ring A is an unsubstituted furyl ring and B is an optional substituted phenyl ring;
and with the proviso that when R¹ and R² are methoxy, R³ is hydrogen, m is 0, R⁴ and R⁵ together are ═O, B is an optional substituted phenyl ring and one of R⁸ or R⁹ is hydrogen the other of R⁸ or R⁹ is not —CH₂CN or optionally substituted forms thereof;
and with the proviso that ring A is not an unsubstituted cyclopentadiene ring, when R¹ and R² are methoxy, R³ is hydrogen, R⁴ and R⁵ together are ═O, R⁶ and R⁸ together form a bond, R⁷ and R⁹ are hydrogen and B is an optionally substituted phenyl or pyridine ring;
and with the proviso that that R³ is not —(CH₂)₂NR′R″ (where R′ and R″ are independently hydrogen or alkyl, or together with the nitrogen to which they are attached form an unsubstituted piperidine ring), when R¹ and R² are methoxy, R⁴ is hydroxy, R⁵, R⁶, R⁷, R⁸ and R⁹ are hydrogen, ring A is a five membered heterocyclic ring containing oxygen, and B is an optionally substituted phenyl ring;
and its salts and pharmaceutically acceptable derivatives thereof.

In an aspect of the invention there is provided a method for the treatment or prevention of autoimmune or chronic inflammatory diseases, or the prevention of rejection of foreign organ transplants and/or related afflictions, by the administration of a compound of formula I or a pharmaceutically acceptable derivative thereof, or a composition containing a compound of formula I or pharmaceutically acceptable derivatives thereof.

In another aspect of the invention there is provided a method of intentionally modulating potassium ion channel activity of T-cells by the application of a compound of Formula I, or a pharmaceutically acceptable derivative thereof, to said T-cells.

In a further aspect of the invention there is provided a pharmaceutical composition for use as an immunosuppressant, the composition comprising an effective amount of compound of Formula I or pharmaceutically acceptable derivative thereof and optionally a carrier or diluent.

In another aspect of the invention there is provided a process for the production of compounds of formula I, its salts and pharmaceutically acceptable derivatives thereof.

### BRIEF DESCRIPTION OF THE DRAWINGS

FIG. 1 depicts the effects [³H]-Thymidine incorporation by human lymphocytes.

### DETAILED DESCRIPTION OF THE INVENTION

The invention is based on the discovery that compounds of the general formula I, as described in the above Summary of the Invention can have useful properties as inhibitors of potassium cell channels, and particularly the Kv1.3 channel. Such compounds have significant potential as immunosuppressants for the treatment of autoimmune disorders such as multiple sclerosis and rheumatoid arthritis. They may also be useful in the treatment or prevention of graft rejection.

The term “alkyl” as used alone or in combination herein refers to a straight or branched chain saturated hydrocarbon group containing from one to ten carbon atoms, preferably one to six carbon atoms. The terms “C₁₋₆ alkyl” and “lower alkyl” refer to such groups containing from one to six carbon atoms, preferably one to four carbon atoms. Preferred alkyl groups include methyl (“Me”), ethyl (“Et”), n-propyl, isopropyl, n-butyl, isobutyl, sec-butyl, tert-butyl and the like.

The term “alkenyl” means a two to ten carbon, preferably two to six carbon, straight or branched hydrocarbon containing one or more double bonds, preferably one or two double bonds. Preferred alkenyl groups include ethenylene, propenylene, 1,3-butadienyl and 1,3,5-hexatrienyl.

The term “alkynyl” means a two to ten carbon, preferably two to six carbon, straight or branched hydrocarbon containing one or more triple bonds, preferably one or two triple bonds.

The term “alkoxy” as used alone or in combination herein refers to a straight or branched chain alkyl group covalently bound via an O linkage and the terms “C₁₋₆ alkoxy” and “lower alkoxy” refer to such groups containing from one to six carbon atoms. Preferred alkoxy and lower alkyl groups are methoxy, ethoxy, propoxy, isopropoxy, butoxy and t-butoxy groups.

The term “aromatic” or “aryl” when used alone or in combination refers to an unsubstituted or optionally substituted monocyclic or bicyclic aromatic hydrocarbon ring system. The preferred aromatic ring are optionally substituted phenyl (“Ph”) or naphthalenyl groups.

The more preferred aromatic or aryl group is the phenyl group which may be optionally substituted with up to five but more usually with one or two optional substituents. The preferred optional substituents include C₁₋₆ alkyl, C₁₋₆ alkoxy, as well as cyano, trifluoromethyl and halo.

The term “benzofused” as used herein refers to a fused polycyclic ring system formed by joining an optionally substituted benzene ring to another ring, in such a way that the two rings share two ring atoms.

The term “carbocyclic” as used herein refers to a stable monocyclic or polycyclic ring system, wherein the ring atoms are only carbon atoms. The rings may be aromatic or non-aromatic. Examples of rings include cyclopentane, cyclohexane and benzene. The carbocyclic ring may be optionally substituted with one or more substituents.

The term “heterocyclic” as used herein refers to a stable monocyclic or polycyclic ring system containing at least one ring of carbon atoms and other atoms selected from nitrogen, sulfur and oxygen. It includes aromatic (including what is sometimes referred to as pseudoaromatic) and non aromatic rings. The term “pseudoaromatic” refers to a ring system which is not strictly aromatic, but which is stabilised by means of delocalisation of electrons and behaves in a similar manner to aromatic rings.

The rings or ring systems generally include 1 to 9 carbon atoms in addition to the heteroatom(s) and may be saturated, unsaturated, aromatic or pseudoaromatic.

Examples of 5-membered monocyclic heterocycles include furyl, thienyl, pyrrolyl, oxazolyl, thiazolyl, isoxazolyl, isothiazolyl, pyrazolyl, imidazolyl, triazolyl, tetrazolyl, oxadiazolyl, thiadiazolyl and examples of 6-membered monocyclic heterocycles include pyridyl, pyrimidinyl, pyridazinyl, pyrazinyl and triazinyl, each of which may be optionally substituted.

The heterocyclic ring may be fused to a carbocyclic ring such as phenyl.

Examples of 9 and 10-membered bicyclic heterocycles include indolyl, benzofuranyl, benzothienyl, benzoxazolyl, benzothiazolyl, benzisoxazolyl, benzisothiazolyl, indazolyl, isoquinolinyl, quinolinyl, quinoxalinyl, cinnolinyl, phthalazinyl, quinazolinyl, benzotriazinyl and the like.

Examples of preferred heterocyclic radicals include (optionally substituted) isoxazolyls, isothiazolyls, 1,3,4-oxadiazolyls, 1,3,4-thiadiazolyls, 1,2,4-oxadiazolyls, 1,2,4-thiadiazolyls, oxazolyls, thiazolyls, pyridinyls, pyridazinyls, pyrimidinyls, pyrazinyls, 1,2,4-triazinyls, 1,3,5-triazinyls, benzoxazolyls, benzothiazolyls, benzisoxazolyls, benzisothiazolyls, quinolinyls and quinoxalinyls.

Examples of unsaturated 5-membered heterocyclic rings include oxazolyl, thiazolyl, imidazolyl, 1,2,3-triazolyl, isoxazolyl, isothiazolyl, pyrazolyl, furyl, thiophenyl and pyrrolyl. Examples of unsaturated 6-membered heterocyclic rings include pyridinyl, pyrimidinyl, pyrazinyl, pyridazinyl and 1,2,4-triazinyl.

In a preferred embodiment, the heterocyclic ring is an aromatic ring selected from the group consisting of furyl, thienyl, pyridyl, purrolyl, oxazolyl, thiazolyl, imidazolyl, pyrazolyl, isoxazolyl, isothiazolyl, 1,2,3-oxadiazolyl, 1,2,4-oxadiazolyl, 1,2,4-oxadiazol-5-one, 1,2,3-triazolyl, 1,3,4-thiadiazolyl, pyridazinyl, pyrimidinyl, pyrazinyl, 1,3,5-triazinyl, indolizinyl, indolyl, isoindolyl, 3H-indolyl, indolinyl, benzo[b]furanyl, benzo[b]thiophenyl, 1H-indazolyl, benzimidazolyl and tetrazolyl.

In another preferred embodiment, the heterocyclic ring is a non-aromatic ring selected from the group consisting of pyrrolidinyl, imidazolinyl, 2-imidazolidonyl, 2-pyrrolidonyl, pyrrolin-2-onyl, tetrahydrofuryl, 1,3-dioxolanyl, piperidinyl, tetrahydropyryl, oxazolinyl, 1,3-dioxanyl, 1,4-piperazinyl, morpholinyl and thiomorpholinyl.

The term “heteroaromatic” as used herein is limited to aromatic (including pseudoaromatic) heterocycles as described above. Preferred rings include 5 or 6-membered monocyclic rings or an 8-11 membered bicyclic rings containing one, two, or three ring heteroatoms selected from nitrogen, oxygen and sulfur.

Examples of preferred heteroaromatic groups include isoxazolyl, oxazolyl, imidazolyl, thiazolyl, isothiazolyl, pyridyl, pyrimidinyl, furyl, pyrazolyl, pyridazinyl, furazanyl and thienyl. The ring may be attached to the parent structure through a carbon atom or through any heteroatom of the heteroaryl that results in a stable structure. Where indicated the heteroaryl may be fused to the parent structure.

The terms “halo” and “halogen” as used herein represent fluorine, chlorine, bromine or iodine substituent moieties, preferably bromine, chlorine or fluorine.

In this specification unless otherwise defined “optionally substituted” means that a group may or may not be further substituted with one or more groups independently selected from:—
cyano, halo, —B(OH)₂, nitro, alkyl, alkenyl, alkynyl, cycloalkyl, aryl and heterocyclyl;
—OR, —C(O)R, —C(O)OR, —OC(O)R, —SR, —SO₂R, —SO₃R, —OSO₃R, —S(O)₂NHC(O)R, —S(O)₂NHS(O)₂R, —PO₃, —OPO₃R₂ and —C(O)NHS(O)₂R (where R is hydrogen, alkyl, alkenyl, alkynyl, cycloalkyl, aryl, heterocyclyl, arylalkyl, arylalkenyl, arylalkynyl or heterocyclylalkyl);
—C(O)NR′R″, —C(S)NR′R″, —C(NR)NR′R″, —C(═NCN)—NR′R″, —C(═NR)NR′R″, —C(═NR′)SR″, —C(S)NR′R″, —NR′C(O)R″, —NR′C(O)OR″, —NRC(O)NR′R″, —NRC(S)NR′R″, —NR′C(O)R″, —NR′C(═NCN)SR″, —NR′SO₂R″ and —NR′C(S)R″ (where R, R′ and R″ are independently selected from hydrogen, alkyl, alkenyl, alkynyl, aryl and heterocyclyl); or
NR′R″ (where R′ and R″ are independently selected from hydrogen, alkyl, alkenyl, alkynyl, aryl, arylalkyl, arylalkenyl, arylalkynyl and alkoxy, or R′ and R″ together with the N atom to which they are attached form a six membered ring);

Where the optional substituent includes an alkyl, alkenyl, alkynyl or cycloalkyl moiety, that moiety may itself be substituted with one or more of groups independently selected from halo, hydroxy, cyano, —B(OH)₂, —OSO₃H, —OPO₃H₂, tetrazolyl, lower alkoxy, —S(O)₂NHC(O)R, —C(O)NHS(O)₂R, —COR, —COOR (where R is hydrogen, lower alkyl or phenyl) and —NR′R″, (where R′, and R″ are independently hydrogen or lower alkyl or R′ and R″ together with the N atom to which they are attached form a six membered ring).

Where the optional substituent includes a carbocyclic or heterocyclic ring, that ring may be substituted at one or more substitutable ring positions with one or more groups independently selected from alkyl (preferably lower alkyl), alkoxy (preferably lower alkoxy), nitro, monoalkylamino (preferably a lower alkylamino), dialkylamino (preferably a di[lower]alkylamino, cyano, halo, haloalkyl (preferably trifluoromethyl), alkanoyl, aminocarbonyl, monoalkylaminocarbonyl, dialkylaminocarbonyl, alkyl amido (preferably lower alkyl amido), alkoxyalkyl (preferably a lower alkoxy[lower]alkyl), alkoxycarbonyl (preferably a lower alkoxycarbonyl), alkylcarbonyloxy (preferably a lower alkylcarbonyloxy) and aryl (preferably phenyl), said aryl being optionally substituted by halo, lower alkyl and lower alkoxy groups.

The salts of the compound of formula I are preferably pharmaceutically acceptable, but it will be appreciated that non-pharmaceutically acceptable salts also fall within the scope of the present invention, since these are useful as intermediates in the preparation of pharmaceutically acceptable salts.

The term “pharmaceutically acceptable derivatives” includes pharmaceutically acceptable esters, prodrugs, solvates and hydrates, and pharmaceutically acceptable addition salts of the compounds or the derivatives. Pharmaceutically acceptable derivatives may include any pharmaceutically acceptable salt, hydrate or any other compound or prodrug which, upon administration to a subject, is capable of providing (directly or indirectly) a compound of formula I or an antivirally active metabolite or residue thereof.

The pharmaceutically acceptable salts include acid addition salts, base addition salts, salts of pharmaceutically acceptable esters and the salts of quaternary amines and pyridiniums. The acid addition salts are formed from a compound of the invention and a pharmaceutically acceptable inorganic or organic acid including but not limited to hydrochloric, hydrobromic, sulfuric, phosphoric, methanesulfonic, toluenesulphonic, benzenesulphonic, acetic, propionic, ascorbic, citric, malonic, fumaric, maleic, lactic, salicyclic, sulfamic, or tartartic acids. The counter ion of quarternary amines and pyridiniums include chloride, bromide, iodide, sulfate, phosphate, methansulfonate, citrate, acetate, malonate, fumarate, sulfamate, and tartate. The base addition salts include but are not limited to salts such as sodium, potassium, calcium, lithium, magnesium, ammonium and alkylammonium. Also, basic nitrogen-containing groups may be quaternised with such agents as lower alkyl halides, such as methyl, ethyl, propyl, and butyl chlorides, bromides and iodides; dialkyl sulfates like dimethyl and diethyl sulfate; and others. The salts may be made in a known manner, for example by treating the compound with an appropriate acid or base in the presence of a suitable solvent.

The compounds of the invention may be in crystalline form or as solvates (e.g. hydrates) and it is intended that both forms be within the scope of the present invention. The term “solvate” is a complex of variable stoichiometry formed by a solute (in this invention, a compound of the invention) and a solvent. Such solvents should not interfere with the biological activity of the solute. Solvents may be, by way of example, water, ethanol or acetic acid. Methods of solvation are generally known within the art.

The term “pro-drug” is used in its broadest sense and encompasses those derivatives that are converted in vivo to the compounds of the invention. Such derivatives would readily occur to those skilled in the art, and include, for example, compounds where a free hydroxy group is converted into an ester derivative or a ring nitrogen atom is converted to an N-oxide. Examples of ester derivatives include alkyl esters, phosphate esters and those formed from amino acids, preferably valine. Any compound that is a prodrug of a compound of the invention is within the scope and spirit of the invention.

The term “pharmaceutically acceptable ester” includes biologically acceptable esters of compound of the invention such as sulphonic, phosphonic and carboxylic acid derivatives.

It will be appreciated that compound of formula I and some derivatives thereof may have at least one asymmetric centre, and therefore are capable of existing in more than one stereoisomeric form. The invention extends to each of these forms individually and to mixtures thereof, including racemates. The isomers may be separated conventionally by chromatographic methods or using a resolving agent. Alternatively the individual isomers may be prepared by asymmetric synthesis using chiral intermediates. Where the compound has at least one carbon-carbon double bond, it may occur in Z- and E-forms and all isomeric forms of the compounds being included in the present invention.

The invention provides a method of preventing or treating autoimmune or chronic inflammatory diseases, or the prevention of rejection of foreign organ transplants and/or related afflictions, by the administration of a compound of formula I, or a pharmaceutically acceptable derivative thereof, or a composition containing a compound of the general formula I or a pharmaceutically acceptable derivative thereof.

With reference to the general formula I, it is preferred that the fused ring A is an optionally substituted ring selected from the following (the two dashed lines on the right hand side of the rings indicate the position at which the ring A is fused to the phenyl ring):— 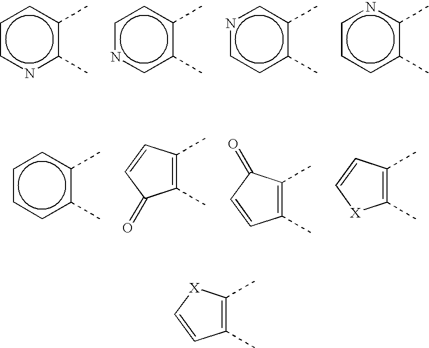
where X is O, S or NR, where R is hydrogen, lower alkyl or oxygen; or 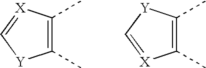
where X is N, and Y is O, S or NR and R is hydrogen, lower alkyl or oxygen.

More preferably ring A is an optionally substituted ring of the structure:— 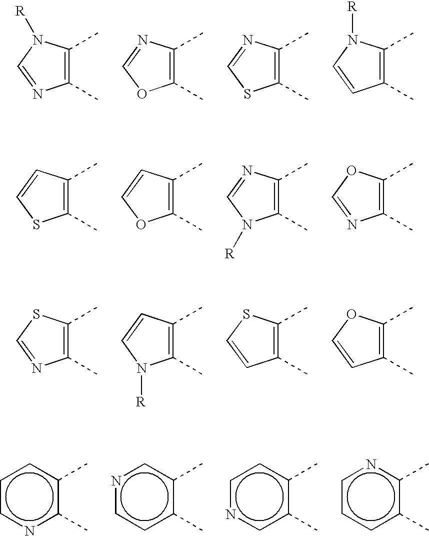
where R is hydrogen or lower alkyl.

Most preferably A is an optionally substituted ring of the structure:—

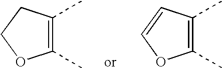

Preferably ring A is optionally substituted with halo, lower alkyl, benzyl or —C(O)C₆H₅.

Preferably R¹ and R² are independently selected from hydrogen; halogen; hydroxy; lower alkoxy, optionally substituted benzyl, optionally substituted phenyl, optionally substituted diphenyl, optionally substituted phenoxy and optionally substituted benzoxy group. More preferably R¹ and R² are independent selected from hydrogen, lower alkoxy, optional substituted benzoxy and optionally substituted phenoxy. Most preferably they are both methoxy groups.

Preferably R³ is hydrogen or optionally substituted lower alkyl, or together with R⁶ form a five or six membered heterocylic ring. If R³ and R⁶ form a heterocyclic ring it is preferred that the ring is not heteroaromatic and that one or more of the ring carbons is substituted with ═O, ═S or ═NR, where R is hydrogen or lower alkyl.

Preferably R³ is selected from hydrogen, unsubstituted alkyl (preferably lower alkyl), —(CH₂)ₙNR′R″ (where n is from 1 to 4 and R′ and R″ are independently hydrogen or lower alkyl, or R′ and R″ together with the N atom to which they are attached form a six membered ring) and —(CH₂)ₙR²⁰, (where n is from 1 to 6 and R²⁰ is selected from phenyl, —OSO₃H, —OPO₃H₂, —CO₂H, tetrazolyl, —B(OH)₂, —CO₂R, —S(O)₂NHC(O)R and —S(O)₂NHS(O)₂R, where R is lower alkyl).

Most preferably R³ is hydrogen, methyl or benzyl optionally substituted with 1 to 3 halo or lower alkyl groups.

Preferably R⁴ and R⁵ are independently hydrogen or hydroxy, or together are ═O. Most preferably R⁴ and R⁵ together are ═O.

Preferably R⁶ is selected from hydrogen, halogen (preferably bromine), —CN, —C(O)R (where R is lower alkyl or phenyl), —C(O)OR, (where R is hydrogen or lower alkyl), optionally substituted alkyl, (such as arylalkyl or —(CH₂)ₙCO₂R, where R is H or methyl and n is from 1 to 6) and optionally substituted alkenyl group (such as phenylethylene); or preferably R⁵ and R⁸ together form a bond between the carbons to which they are attached.

Preferably R⁷ is hydrogen.

Preferably R⁸ and R⁹ are independently selected from hydrogen; lower alkyl, an optionally substituted cyanoalkyl group (such as —CHR(CN) where R is selected from hydrogen, OH, lower alkyl and lower alkoxy), —C(O)R (where R is optionally substituted lower alkyl, optionally substituted lower alkoxy or optionally substituted phenyl), —NR′R″ (where R′ and R″ are independently selected from hydrogen and lower alkyl), and

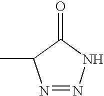

More preferably R⁸ together with R⁶ form a carbon double bond, and R⁹ is hydrogen.

Preferably m is 0 or 1, most preferably 0.

Preferably B is an optionally substituted phenyl ring. This ring may also be benzofused or fused to a heterocyclic ring. Preferred forms of B include an optionally substituted phenyl or naphthalene ring, or a ring system of the structure C

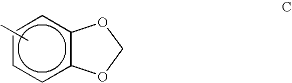

Alternatively B is an optionally substituted and optionally benzofused heteroaromatic ring. Preferred heteroaromatic rings include pyrrole, furan, thiophene, imidazole, pyrazole, thiazole, oxazole, pyridine, pyran and pyrimidine. When B is a benzofused heteroaromatic ring, it is preferrably an optionally substituted indole, quinoline or isoquinoline ring system.

In addition to the above forms of B, R⁶, R⁷ and R⁸ together with a ring carbon atom of Ring B can form a six membered aromatic ring fused to ring B to provide a compound of the following general formula:—

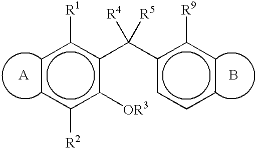

Preferably B is a phenyl ring optionally substituted with one or more substituents independently selected from
halo, cyano, —NO₂, —SO₃, —OSO₃H, —OPO₃H₂, —PO₃ and —B(OH)₂;
—NR′R″ (where R′ and R″ are independently hydrogen or lower alkyl);
—NR′C(O)R″ (where R′ and R″ are independently hydrogen or lower alkyl);
phenyl and tetrazolyl;
—OR, —C(O)R, and —C(O)OR (where R is hydrogen, optionally substituted lower alkyl, optionally substituted phenyl, optionally substituted phenylloweralkyl (where the optional substituents are independently selected from lower alkyl, halo and —NR′R″ where R′ and R″ are independently hydrogen or lower alkyl);
—C(O)NHSO₂R′″ and —S(O)₂NHC(O)R′″ (where R′″ is lower alkyl);
optionally substituted lower alkyl such as —CH₃, —CH(CH₃)₂, —CH₂B(OH)₂, —CH₂PO₃, —CH₂SO₃, —CH₂OPO₃H₂, —CH₂OSO₃H, —CH₂C(O)NHSO₂R′″, —CH₂S(O)₂NHC(O)R′″ (where R′″ is lower alkyl), —CH₂C₆H₅, —CH₂-tetrazolyl, —(CH₂)ₙNR′R″ (where n is from 1 to 4 and R′ and R″ are independently hydrogen or lower alkyl); —CF₃, —CF₂B(OH)₂, —CF₂PO₃, —CF₂SO₃, —CF₂OPO₃H₂, —CF₂OSO₃H, —CF₂C(O)NHSO₂R′″, —CF₂S(O)₂NHC(O)R′″ (where R′″ is lower alkyl) —CF₂C₆H₅ and —CF₂-tetrazolyl.

In a preferred form of the invention, B is meta substituted (in respect to the bond that joins B to the rest of the general formula) with an acidic group. Non limiting examples of acidic groups include —(CH₂)ₙR²⁰, where n is from 0 to 6, and R²⁰ is selected from —OSO₃H, —OPO₃H₂, —CO₂H, tetrazolyl, —B(OH)₂, —S(O)₂NHC(O)R′, —C(O)NHS(O)₂R′ (where R′ is lower alkyl), —OH, —C₆H₄OH, —CF₂PO₃ and —SO₃, most preferably B is substituted with one or more hydroxy groups. B may also have one or more additional substituents.

A preferred form of the invention pertains to the use of compounds of formula II for preventing or treating autoimmune or chronic inflammatory diseases, or the prevention of rejection of foreign organ transplants and/or related afflictions. 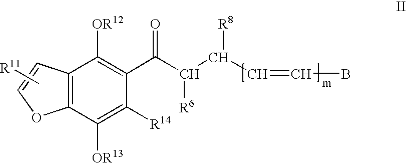
where B is as earlier described, m is 0 or 1, and R⁶ and R⁸ are hydrogen or together form a double bond, and R¹¹ is hydrogen, lower alkyl, halogen and —C(O)C₆H₅, R¹² and R¹³ are independently selected from hydrogen, alkyl, optionally substituted phenyl, optionally substituted benzyl, —(CH₂)ₙNR′R″ (where n is from 1 to 4 and R′ and R″ are independently hydrogen or lower alkyl) and —(CH₂)ₙR²⁰, where n is from 1 to 4, and R²⁰ is selected from —OSO₃H, —OPO₃H₂, —CO₂H, tetrazolyl, —B(OH)₂, —S(O)₂NHC(O)R and —C(O)NHS(O)₂R (where R is lower alkyl) and R¹⁴ is hydroxy or alkoxy, preferably hydroxy or methoxy.

A more preferred form of the invention is the use of compounds of the formula III for preventing or treating autoimmune or chronic inflammatory diseases, or the prevention of rejection of foreign organ transplants and/or related afflictions. 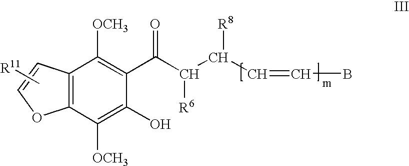
where B is as earlier described, m is 0 or 1, and R⁶ and R⁸ are hydrogen or together form a bond, and R¹¹ is hydrogen, lower alkyl, halogen or —C(O)C₆H₅.

A more preferred form of the invention is the use of compounds of the formula IV for preventing or treating autoimmune or chronic inflammatory diseases, or the prevention of rejection of foreign organ transplants and/or related afflictions. 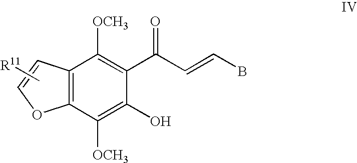
where B is an optionally substituted ring or ring system selected from phenyl, naphthalenyl, pyridinyl, pyrrolyl, furyl, indolyl, quinolinyl, isoquinolinyl, thiophenyl and 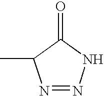
all of which may be optionally substituted with one or more substituents.

The optional substituents of B are preferably independently selected from —OPO₃H₂, —PO₃, —OSO₃, —SO₃, —CH₂PO₃, —CH₂SO₃, —CO₂H, —CH₂CO₂H, —CF₂PO₃, —CF₂SO₃, —OH, —B(OH)₂, —OCH₃, —OCH₂CH₃, —CF₃, —CH₃, —CH₂CH₃, —CH(CH₃)₂, —C₆H₅, —OC₆H₅—OC₆H₄CH₃, -tetrazolyl, —CH₂tetrazolyl, —CF₂tetrazolyl, —NHC(O)CH₃, —F, —Cl, —Br, —CN, —OCH₂CH₂N(CH₂CH₃)₂, —NO₂, —N(CH₃)₂ and

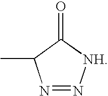

Another preferred form of the invention is the use of compounds of the formula V for preventing or treating autoimmune or chronic inflammatory diseases, or the prevention of rejection of foreign organ transplants and/or related afflictions. 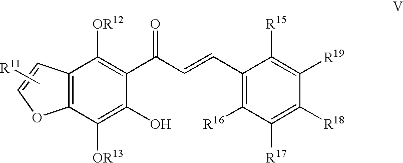
where R¹¹ is hydrogen, lower alkyl, halogen or —C(O)C₆H₅R¹² preferably hydrogen, and R¹³ are independently selected from hydrogen, alkyl (preferably lower alkyl), optionally substituted phenyl and optionally substituted benzyl; R¹³ also be selected from —(CH₂)ₙNR′R″ (where n is from 1 to 4 and R′ and R″ are independently selected from hydrogen and lower alkyl) and —(CH₂)ₙR²⁰ (where n is from 0 to 6 and R²⁰ is selected from —OSO₃H, —OPO₃H₂, —CO₂H, -tetrazolyl, —B(OH)₂, —S(O)₂NHC(O)R and —C(O)NHS(O)₂R where R is lower alkyl).

R¹⁵, R¹⁶, R¹⁷ and R¹⁸ are independently selected from hydrogen, —OPO₃H₂, —PO₃, —OSO₃, —SO₃, —CH₂PO₃, —CH₂SO₃, —CO₂H, —CH₂CO₂H, —CF₂PO₃, —CF₂SO₃, —OH, —B(OH)₂, —OCH₃, —OCH₂CH₃, —CF₃, —CH₃, —CH₂CH₃, —CH(CH₃)₂, —C₆H₅, —OC₆H₅—OC₆H₄CH₃, -tetrazolyl, —CH₂tetrazolyl, —CF₂tetrazolyl, —NHC(O)CH₃, —F, —Cl, —Br, —CN, —OCH₂CH₂N(CH₂CH₃)₂, —NO₂, —N(CH₃) and 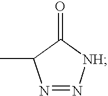
R¹⁹ is selected from —(CH₂),R²⁰, where n is from 0 to 6, and R²⁰ is selected from hydrogen (when n is other than 0), —OSO₃H, —OPO₃H₂, —CO₂H, -tetrazolyl, —B(OH)₂, —S(O)₂NHC(O)R′, —C(O)NHS(O)₂R′, —OR (where R′ is lower alkyl), —R—C₆H₄OH, —CF₂PO₃ and —SO₃. Preferably R¹⁹ is hydroxy.

Another preferred form of the invention is the use of compounds of the formula VI for preventing or treating autoimmune or chronic inflammatory diseases, or the prevention of rejection of foreign organ transplants and/or related afflictions. 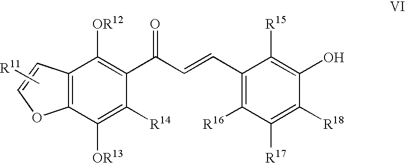
where R¹¹ is hydrogen, lower alkyl, halogen or —C(O)C₆H₅, preferably hydrogen;
R¹² and R¹³ are independently selected from hydrogen, alkyl (preferably lower alkyl), optionally substituted phenyl and optionally substituted benzyl;
and R¹³ may also be selected from —(CH₂)ₙNR′R″ (where n is from 1 to 4 and R′ and R″ are independently hydrogen or lower alkyl) or —(CH₂)ₙR²⁰, where n is from 0 to 6, and R²⁰ is selected from —OSO₃H, —OPO₃H₂, —CO₂H, tetrazolyl, —B(OH)₂, —S(O)₂NHC(O)R and —C(O)NHS(O)₂R where R is lower alkyl);
R¹⁴ is hydroxy, alkoxy, —(CH₂)ₙNR′R″ (where n is from 1 to 4 and R′ and R″ are independently hydrogen or lower alkyl) and —(CH₂)ₙR²⁰, where R²⁰ is selected from —OSO₃H, —OPO₃H₂, —CO₂H, tetrazolyl, —B(OH)₂, —S(O)₂NHC(O)R and —S(O)₂NHS(O)₂R where R is lower alkyl. Preferably R¹⁴ is hydroxy or methoxy.

R¹⁵, R¹⁶, R¹⁷ and R¹⁸ are independently selected from hydrogen, —OPO₃H₂, —PO₃, —OSO₃, —SO₃, —CH₂PO₃, —CH₂SO₃, —CO₂H, —CH₂CO₂H, —CF₂PO₃, —CF₂SO₃, —OH, —B(OH)₂, —OCH₃, —OCH₂CH₃, —CF₃, —CH₃, —CH₂CH₃, —CH(CH₃)₂, —C₆H₅, —OC₆H₅—OC₆H₄CH₃, -tetrazolyl, —CH₂tetrazolyl, —CF₂tetrazolyl, —NHC(O)CH₃, —F, —Cl, —Br, —CN, —OCH₂CH₂N(CH₂CH₃)₂, —NO₂, —N(CH₃) and

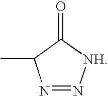

The compounds of formula I to VI, pharmaceutically acceptable derivatives thereof, and compositions thereof, may be useful in the treatment of autoimmune diseases, the prevention of rejection of foreign organ transplants and/or related afflictions, diseases and illnesses.

The potassium channel activity inhibited by the compounds of Formula I to VI is may be a voltage-gated potassium channel, for example, Kv1.1-Kv1.7, or heteromultimers containing these proteins and/or accessory proteins such as beta subunits.

Compounds of the Formula I to VI may inhibit the potassium ion channel activity of the voltage-gated potassium channel, Kv1.3 channel of a T-cell.

The compounds of the invention may be useful in respect of a number of ailments. They may be useful in the therapeutic or prophylactic treatment of the resistance to transplantation of organs or tissue (such as heart, kidney, liver, lung, bone marrow, cornea, pancreas, intestinum tenue, limb, muscle, nervus, medulla ossium, duodenum, small-bowel, medulla ossium, skin, pancreatic islet-cell, etc. including xeno transplantation), graft-versus-host diseases; rheumatoid arthritis, systemic lupus erythematosus, nephrotic syndrome lupus, Palmo-planter pustulosis, Hashimoto's thyroiditis, multiple sclerosis, Guillain-Barre syndrome, myasthenia gravis, type I diabetes uveitis, juvenile-onset or recent-onset diabetes mellitus, diabetic neuropathy, posterior uveitis, allergic encephalomyelitis, glomerulonephritis, infectious diseases caused by pathogenic microorganisms, inflammatory and hyperproliferative skin diseases, psoriasis, atopical dermatitis, contact dermatitis, eczematous dermatitises, seborrhoeis dermatitis, Lichen planus, Pemphigus, bullous pemphigoid, Epidermolysis bullosa, urticaria, angioedemas, vasculitides, erythemas, cutaneous eosinophilias, Lupus erythematosus, acne, Alopecia greata, keratoconjunctivitis, vernal conjunctivitis, uveitis associated with Behcet's disease, keratitis, herpetic keratitis, conical cornea, dystrophia epithelialis corneae, corneal leukoma, ocular pemphigus, Mooren's ulcer, Scleritis, Graves' opthalmopathy, Vogt-Koyanagi-Harada syndrome, sarcoidosis, etc.; pollen allergies, reversible obstructive airway disease, bronchial asthma, allergic asthma, intrinsic asthma, extrinsic asthma and dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns and leukotriene B₄-mediated diseases, Coeliac diseases, proctitis, eosinophilic gastroenteritis, mastocytosis, Crohn's disease, ulcerative colitis, migraine, rhinitis, eczema, interstitial nephritis, Good-pasture's syndrome, hemolytic-uremic syndrome, diabetic nephropathy, multiple myositis, Guillain-Barre syndrome, Meniere's disease, polyneuritis, multiple neuritis, mononeuritis, radiculopathy, hyperthyroidism, Basedow's disease, pure red cell aplasia, aplastic anemia, hypoplastic anemia, idiopathic thrombocytopenic purpura, autoimmune hemolytic anemia, agranulocytosis, pernicious anemia, megaloblastic anemia, anerythroplasia, osteoporosis, sarcoidosis, fibroid lung, idiopathic interstitial pneumonia, dermatomyositis, leukoderma vulgaris, ichthyosis vulgaris, photoallergic sensitivity, cutaneous T-cell lymphoma, arteriosclerosis, atherosclerosis, aortitis syndrome, polyarteritis nodosa, myocardosis, scleroderma, Wegener's granuloma, Sjogren's syndrome, adiposis, eosinophilic fascitis, lesions of gingiva, periodontium, alveolar bone, substantia ossea dentis, glomerulonephritis, male pattern alopecia or alopecia senilis by preventing epilation or providing hair germination and/or promoting hair generation and hair growth; muscular dystrophy; Pyoderma and Sezary's syndrome, Sjoegren's syndrome, Addison's disease, ischemia-reperfusion injury of organs which occurs upon preservation, transplantation or ischemic disease, for example, thrombosis and cardiac infraction, endotoxin-shock, pseudomembranous colitis, colitis caused by drug or radiation, ischemic acute renal insufficiency, chronic renal insufficiency, toxinosis caused by lung-oxygen or drug, for example, paracort and bleomycins, lung cancer, pulmonary emphysema, cataracta, siderosis, retinitis, pigmentosa, senile macular degeneration, vitreal scarring, corneal alkali burn; dermatitis erythema multiforme, linear IgA ballous dermatitis and cement dermatitis, gingivitis, periodontitis, sepsis, pancreatitis, diseases caused by environmental pollution, aging, carcinogenis, metastasis of carcinoma and hypobaropathy; disease caused by histamine or leukotriene-C₄ release; Berger's disease, Behcet's disease, autoimmune hepatitis, primary biliary cirrhosis sclerosing cholangitis, partial liver resection, acute liver necrosis, necrosis caused by toxin, viral hepatitis, shock, or anoxia, B-virus hepatitis, non-A/non-B hepatitis, cirrhosis, alcoholic cirrhosis, hepatic failure, fulminant hepatic failure, late-onset hepatic failure, “acute-on-chronic” liver failure, augmentation of chemotherapeutic effect, preventing or treating activity of cytomegalovirus infection, HCMV infection, and antiinflammatory activity; and treatment of immunodepression or a disorder involving immunodepression, including AIDS, cancer, senile dementia, trauma, chronic bacterial infection, type II diabetes mellitus as glucose-dependent insulin secretagogues, cardiac arrhythmias such as atrial or ventricular fibrillation, epilepsy, muscular fasciculations, urinary incontinence, certain central nervous system disorders via modulating neural conduction or neurotransmitter release.

For certain of the above mentioned conditions it is clear that the compounds may be used prophylactically as well as for the alleviation of acute symptoms. References herein to “treatment” or the like are to be understood to include such prophylactic treatment, as well as treatment of acute conditions.

In another aspect, the invention provides a method of modulating potassium ion channel activity of T cells by the application of a compound according to Formula I to VI to said T cells.

The compounds of the invention, pharmaceutically acceptable derivatives thereof, and compositions containing the compounds or pharmaceutically acceptable derivatives thereof, may also be used in the treatment of autoimmune diseases, in the prevention of rejection of foreign organ transplants and/or related afflictions, diseases and illnesses.

In such treatment it is preferred that the potassium channel activity inhibited by the compound of Formula I to VI is a voltage-gated potassium channel, for example, Kv1.1-Kv1.7. More preferably the potassium ion channel activity is the voltage-gated potassium channel, Kv1.3 of a T-cell. Preferably the compound selectively inhibits the Kv1.3 channel, and optionally also the Kv1.1 and/or Kv1.2 channels.

In a further aspect of the invention there is provided a pharmaceutical composition for use as an immunosuppressant, the composition comprising an effective amount of compound of Formula I, pharmaceutically acceptable derivative thereof, and optionally a carrier or diluent.

The compositions of this aspect of the invention may further contain one or more other immunosuppressive compounds. For example the compositions may contain a second immunosuppressive agent such as azathioprine, brequinar sodium, deoxyspergualin, mizaribine, mycophenolic acid morpholino ester, cyclosporin, FK-506 and rapamycin.

By “composition” is intended to include the formulation of an active ingredient (the active being at least one compound of the invention or a pharmaceutically acceptable derivative thereof) with encapsulating material as carrier, to give a capsule in which the active ingredient (with or without other carrier) is surrounded by carriers.

The pharmaceutical compositions or formulations include those suitable for oral, rectal, nasal, topical (including buccal and sub-lingual), vaginal or parenteral (including intramuscular, sub-cutaneous and intravenous) administration or in a form suitable for administration by inhalation or insufflation.

The compounds of the invention, together with a conventional adjuvant, carrier, or diluent, may thus be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, in the form of suppositories for rectal administration; or in the form of sterile injectable solutions for parenteral (including subcutaneous) use.

Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed. Formulations containing ten (10) milligrams of active ingredient or, more broadly, 0.1 to one hundred (100) milligrams, per tablet, are accordingly suitable representative unit dosage forms.

The compounds of the present invention can be administrated in a wide variety of oral and parenteral dosage forms. It will be obvious to those skilled in the art that the following dosage forms may comprise, as the active component, either a compound of the invention or a pharmaceutically acceptable salt of a compound of the invention.

For preparing pharmaceutical compositions from the compounds of the present invention, pharmaceutically acceptable carriers can be either solid or liquid. Solid form preparations include powders, tablets, pills, capsules, cachets, suppositories, and dispensable granules. A solid carrier can be one or more substances which may also act as diluents, flavouring agents, solubilisers, lubricants, suspending agents, binders, preservatives, tablet disintegrating agents, or an encapsulating material.

In powders, the carrier is a finely divided solid that is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as admixture of fatty acid glycerides or cocoa butter, is first melted and the active component is dispersed homogeneously therein, as by stirring. The molten homogenous mixture is then poured into convenient sized moulds, allowed to cool, and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution.

Sterile liquid form compositions include sterile solutions, suspensions, emulsions, syrups and elixirs. The active ingredient can be dissolved or suspended in a pharmaceutically acceptable carrier, such as sterile water, sterile organic solvent or a mixture of both.

The compositions according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilisation from solution, for constitution with a suitable vehicle, eg. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavours, stabilisers, buffers, artificial and natural sweeteners, dispersants, thickeners, solubilising agents, and the like.

For topical administration to the epidermis the compounds according to the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Formulations suitable for topical administration in the mouth include lozenges comprising active agent in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multidose form. In the latter case of a dropper or pipette, this may be achieved by the patient administering an appropriate, predetermined volume of the solution or suspension. In the case of a spray, this may be achieved for example by means of a metering atomising spray pump. To improve nasal delivery and retention the compounds according to the invention may be encapsulated with cyclodextrins, or formulated with other agents expected to enhance delivery and retention in the nasal mucosa.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In formulations intended for administration to the respiratory tract, including intranasal formulations, the compound will generally have a small particle size for example of the order of 5 to 10 microns or less. Such a particle size may be obtained by means known in the art, for example by micronisation.

When desired, formulations adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The invention also includes the compounds in the absence of carrier where the compounds are in unit dosage form.

The amount of compound of formula I administered may be in the range from about 10 mg to 2000 mg per day, depending on the activity of the compound and the disease to be treated.

Liquids or powders for intranasal administration, tablets or capsules for oral administration and liquids for intravenous administration are the preferred compositions.

In a further aspect of the invention there is provided new compounds of the general formula I to VI as described above. The compounds of the general formula V and VI are particularly preferred.

In further aspect of the invention there is provided a process for the production of the compounds of the formula I to VI, and more preferably of the formula V and VI.

Chalcones are conveniently synthesized by reaction of an acetophenone with an aryl aldehyde. A useful source of benzofuran-containing acetophenones is natural products such as khellinone.

For example, reaction of khellinone with benzaldehyde in aqueous sodium hydroxide solution furnishes the compound, as shown below:

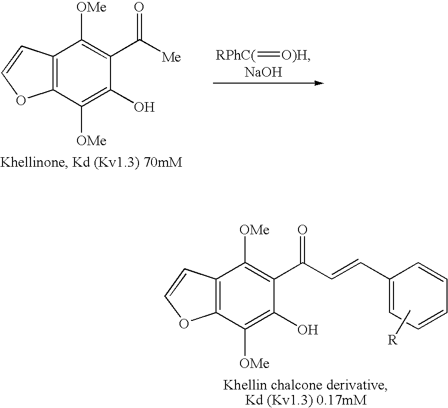

Variations of this reaction include first modifying khellinone to create a derivative thereof by adding, removing or modifying one or more of the functional groups attached to the ring system. For example, the methoxy groups could be selectively manipulated to provide to higher alkyl derivatives of khellinone and used in the above scheme as precursors for compound formation.

Another starting material is Khellin, which can be regarded as a protected khellinone. This compound could be demethylated and the resulting hydroquinone selectively alkylated. As can be seen below hydrogen bonding shown as dotted line will stabilise the hydrogen of one of the phenolic hydroxy groups. A weak base together with an alkylating agent such as Mel or Etl will only alkylate the non-hydrogen bonded phenolic hydroxy group. A strong base, such as Cs₂CO₃, is required together with an alkylating agent such as Mel or Etl to selectively alkylate the hydrogen-bonded phenolic OH.

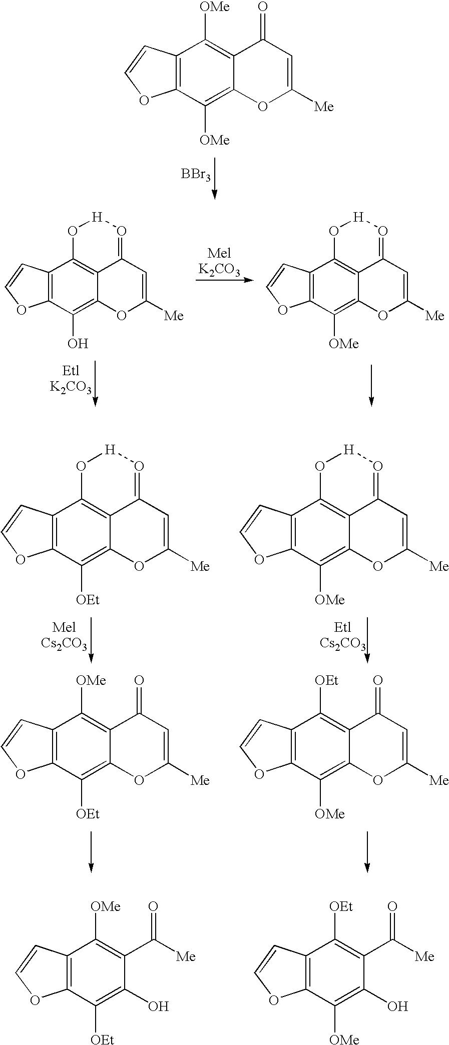

These modified khellinones could then be reacted to give chalcones in the usual way.

Another variation is to add, remove or modify the substituents of the product to form new derivatives. This could be achieved by using standard techniques for functional group inter-conversion, well known in the industry such as those described in Comprehensive organic transformations: a guide to functional group preparations by Larock R C, New York, VCH Publishers, Inc. 1989.

Examples of functional group inter-conversions are: —C(O)NRR′ from —CO₂CH₃ by heating with or without catalytic metal cyanide, e.g. NaCN, and HNRR′ in CH₃OH; —OC(O)R from —OH with e.g., ClC(O)R′ in pyridine; —NR—C(S)NR′R″ from —NHR with an alkylisothiocyanate or thiocyanic acid; —NRC(O)OR from —NHR with alkyl chloroformate; —NRC(O)NR′R″ from —NHR by treatment with an isocyanate, e.g. HN═C═O or RN═C═O; —NRC(O)R′ from —NHR by treatment with ClC(O)R′ in pyridine; —C(═NR)NR′R″ from —C(NR′R″)SR′″ with H₃NR⁺OAc⁻ by heating in alcohol; —C(NR′R″)SR from —C(S)NR′R″ with R—I in an inert solvent, e.g. acetone; —C(S)NR′R″ (where R′ or R″ is not hydrogen) from —C(S)NH₂ with HNR′R″; —C(═NCN)—NR′R″ from —C(═NR′R″)—SR with NH₂CN by heating in anhydrous alcohol, alternatively from —C(═NH)—NR′R″ by treatment with BrCN and NaOEt in EtOH; —NR—C(═NCN)SR from —NHR′ by treatment with (RS)₂C═NCN; —NR″SO₂R from —NHR′ by treatment with ClSO₂R by heating in pyridine; —NR′C(S)R from —NR′C(O)R by treatment with Lawesson's reagent [2,4-bis(4-methoxyphenyl)-1,3,2,4-dithiadiphosphetane-2,4-disulfide]; —NRSO₂CF₃ from —NHR with triflic anhydride and base, —CH(NH₂)CHO from —CH(NH₂)C(O)OR′ with Na(Hg) and HCl/EtOH; —CH₂C(O)OH from —C(O)OH by treatment with SOCl₂ then CH₂N₂ then H₂O/Ag₂O; —C(O)OH from —CH₂C(O)OCH₃ by treatment with PhMgX/HX then acetic anhydride then CrO₃; R—OC(O)R′ from RC(O)R′ by R″CO₃H; —CCH₂OH from —C(O)OR′ with Na/R′OH; —CHCH₂ from —CH₂CH₂OH by the Chugaev reaction; —NH₂ from —C(O)OH by the Curtius reaction; —NH₂ from —C(O)NHOH with TsCl/base then H₂O; —CHC(O)CHR from —CHCHOHCHR by using the Dess-Martin Periodinane regent or CrO₃/aqH₂SO₄/acetone; —C₆H₅CHO from —C₆H₅CH₃ with CrO₂Cl₂; —CHO from —CN with SnCl₂/HCl; —CN from —C(O)NHR with PCl₅; —CH₂R from —C(O)R with N₂H₄/KOH.

Functional group inter-conversion reactions may require other substituents to be protected during the reaction. Suitable protecting groups are well known in industry and have been described in many references such as Protecting Groups in Organic Synthesis, Greene T W, Wiley-Interscience, New York, 1981.

In order that the present invention may be more readily understood we provide the following examples.

### EXAMPLE 1

Khellinone (1 mmol) and benzaldehyde (1.5 mmol) were stirred in 2M aq. NaOH (1 ml) overnight. The reaction mixture was diluted methanol (“MeOH”) (3 ml), acidified with 10% aq. citric acid and the precipitated product filtered and recrystallized from methanol to give the product as cinnamon needles (325 mg, 78%).

### EXAMPLE 2

The product of Example 1 (0.15 mmol) in dichloromethane (“DCM”) (1 ml) was treated with Et₃SiH (2 eq.) and trifluoroacetic acid (“TFA”) (1 mmol), and stirred for 3 h under an atmosphere of dry nitrogen. The reaction mixture was diluted with cyclohexane, and on concentrating, the product crystallised out as yellow needles, which were then filtered off (46 mg, 93%).

### EXAMPLE 3

A suspension of the product of example 1 (0.5 mmol) and 10% Pd/C (60 mg) in ethylacetate (“EtOAc”) (3 ml) was subjected to hydrogenation by balloon overnight. The reaction mixture was filtered through celite, the filtrate concentrated, and the product recrystallized from MeOH as pale yellow needles (103 mg, 63%).

### EXAMPLE 4 TO 58

These were all made by a similar procedure to that described for Example 1, that is, by the reaction of khellinone with an aldehyde. Thus, khellinone (0.4 mmol) and the appropriate aldehyde (0.6 mmol) or an appropriate derivative thereof were stirred in 2M aq. NaOH (1 ml) and MeOH (1 ml) overnight. The reaction mixture was neutralised with acetic acid and the precipitated product filtered and recrystallised from DCM/MeOH.

Noteworthy variations include:

### EXAMPLES 13, 20 AND 40

These were crystallised from DCM/hexane instead of DCM/MeOH.

### EXAMPLES 12 AND 49

These remained as oils.

### EXAMPLES 18, 19, 41 AND 43

These required extended heating and reaction time (up to three days).

In some examples function group interconversion reactions provided the depicted compounds.

### EXAMPLE 59

To the product of Example 1 (0.1 mmol) and Cs₂CO₃ (0.2 mmol) in DMF (0.5 ml) was added Mel (5 equivalents) and the mixture was stirred for 30 minutes, during which time the reaction mixture had changed from a deep red-black to a pale orange colour. The reaction mixture was partitioned between EtOAc (5 ml) and water (5 ml), the separated organic layer washed with 1M aq. NaOH (2×5 ml) and then water (2×5 ml). The organic layer was dried over MgSO₄.H₂O, filtered and the solvent evaporated under vacuum to give the product, which was purified using silica gel chromatography (cyclohexane/DCM). Yield 66%.

### EXAMPLE 60

This was made and purified exactly as for Example 59 but using benzyl bromide (1 equivalent) instead of methyl iodide as the alkylating agent. Yield 73%.

Shown in Table 1 are melting point and biological data for a range of compounds of the invention tested for binding Kv1.3. Those compounds less or not active at Kv1.3 are of interest as being potentially selective for Kv channels other than Kv1.3. They also may be useful intermediates for the manufacture of compounds having activity at the Kv1.3 channel.

TABLE 1

| | | | Est. Kd | |
|---|---|---|---|---|
| | | | (Kv 1.3 unless | OTHER |
| EXAMPLE | | | specified | (melting |
| NO. | STRUCTURE | MW | otherwise) | points ° C.) |
| 1 | 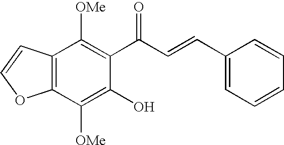 | 324 | Kv1.3 K_{d peak} = 750 nM K_{d end} = 120 nM K_{d area} = 400 nM Kv1.7: K_{d peak} = 25 μM, K_{d end} = 5 μM (i.e. phasic) Kv1.1 K_{d peak} = 12 μM K_{d end} = 700 nM K_{d area} = 1.2 μM IK-no inhibition at 20 μM | Cinnamon needles. Selectivity over Kv1.5 is 25-fold. Mp 125-126 |
| 2 | 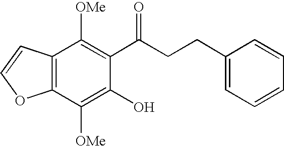 | 326 | K_{d peak} = 800 nM K_{d end} = 300 nM | yellow needles Mp 122-113 |
| 3 | 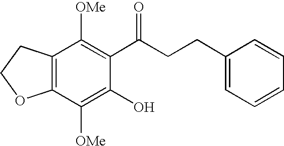 | 328 | K_{d peak} = 2 μM | Pale yellow needles Mp 113-114 |
| 4 | 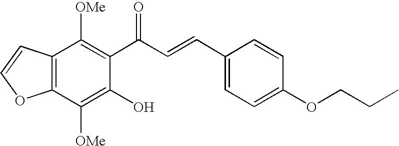 | 382 | K_{d peak} = 45 μM Block not phasic | Mp 74-75 Granular Orange needles |
| 5 | 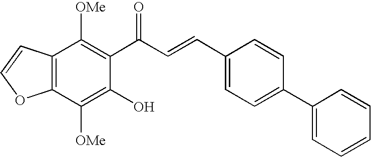 | 400 | K_{d peak} = 30 μM Block not phasic | Mp 122-124 |
| 6 | 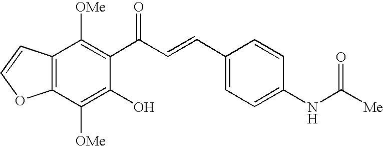 | 381 | K_{d peak} = 35 μM Block not phasic | Mp 180-181 Granular brown solid |
| 7 | 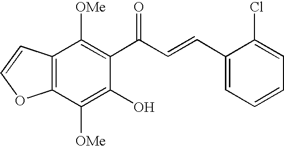 | 358.5 | K_{d peak} = 12 μM Block not phasic | Mp 160-161 Dark orange needles |
| 8 | 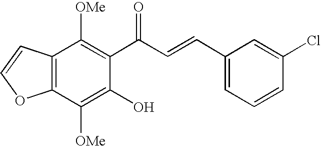 | 358.5 | K_{d peak} = 15 μM Block not phasic | Mp 121 Bright orange needles |
| 9 | 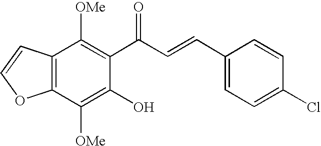 | 358.5 | K_{d peak} = 7 μM Block not phasic | Mp 152 Orange needles |
| 10 | 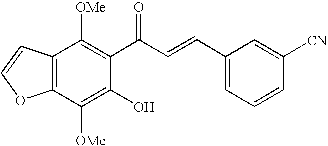 | 349 | K_{d peak} = 20 μM Block not phasic | Mp 182-183 Orange granules |
| 11 | 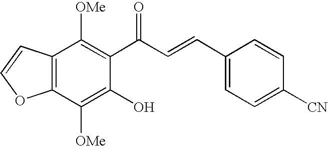 | 349 | K_{d peak} = 12 μM Block not phasic | Mp 196-198 Red-brown solid |
| 12 | 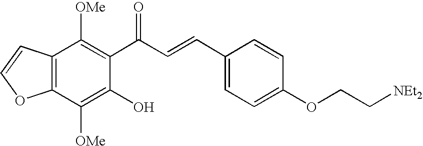 | 439 | 10 μM no effect Not tested against other channels | Dark brown amorphous resin |
| 13 | 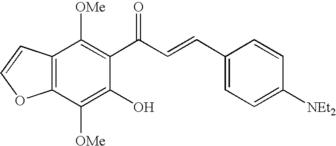 | 395 | K_{d peak} = 18 μM Block not phasic | Mp 95 Red-orange needles |
| 14 | 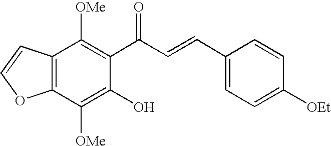 | 368 | K_{d peak} = 10 μM K_{d end} = 1.5 μM | Mp 105 Dark orange granules |
| 15 | 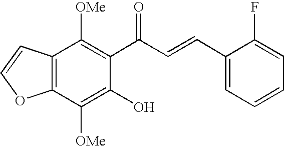 | 342 | K_{d peak} = 90 μM K_{d end} = 12 μM | Mp 133 Orange solid |
| 16 | 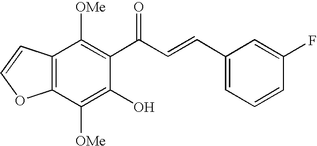 | 342 | K_{d peak} = 35 μM K_{d end} = 4 μM | Mp 121-123 Orange solid |
| 17 | 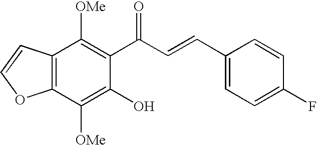 | 342 | K_{d peak} = 8 μM K_{d end} = 1 μM | Mp 129 Orange solid |
| 18 | 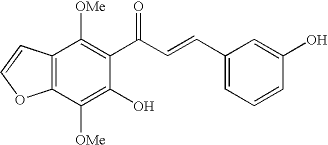 | 340 | Kv1.3- K_{d peak} = 5 μM K_{d end} = 250 nM K_{d area} = 700 nm Kv1.5- K_{d peak} = 16 μM K_{d end} = 10 μM Kv1.7- K_{d} = 50 μM (time- independent) Kv1.1- K_{d peak} = 15 μM K_{d end} = 1 μM K_{d area} = 1.7 μM IK-NI (5 μM) | |
| 19 | 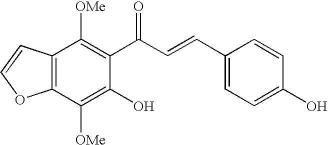 | 340 | K_{d peak} = 10 μM K_{d end} = 0.9 μM | |
| 20 | 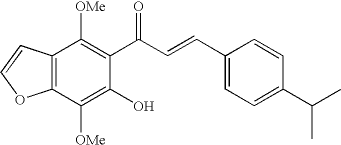 | 366 | K_{d peak} = 3 μM block not phasic | Mp 85 Dark brown crystals |
| 21 | 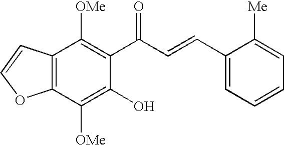 | 338 | K_{d peak} = 2 μM block not phasic | Mp 121 Red-orange powder |
| 22 | 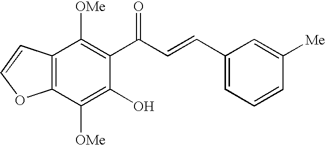 | 338 | K_{d peak} = 1.5 μM K_{d end} = 300 nM | Mp 97-98 Dark brown crystals |
| 23 | 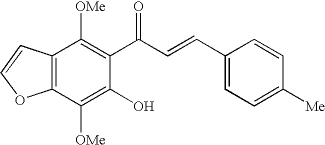 | 338 | K_{d peak} = 1.5 μM K_{d end} = 100 nM | Mp 148 Brown needles |
| 24 | 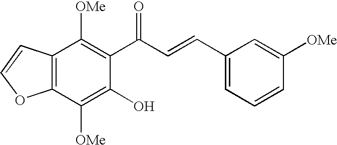 | 354 | Kv1.3- K_{d peak} = 0.9 to 1.1 μM k_{d end} = 250 to 300 nM K_{d area} = 800 nM (based on peak 1.1 and end 300 result) Kv1.5- K_{d peak} = 36 μM K_{d end} = 6 μM IK-NI (20 μM) | Mp 99 Dark orange needles |
| 25 | 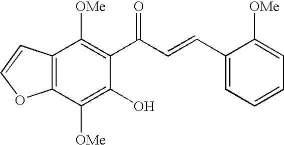 | 354 | K_{d peak} = 5 μM K_{d end} = 1 μM | Mp 117-118 Red-brown granular crystals |
| 26 | 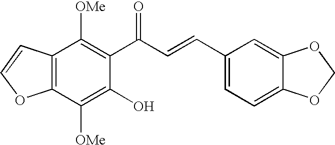 | 398 | K_{d peak} = 9 μM K_{d end} = 1.5 μM | Mp 139-140 Dark orange granular crystals |
| 27 | 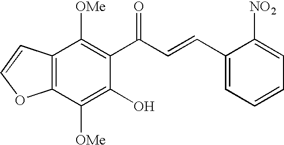 | 369 | K_{d peak} = 15 μM K_{d end} = 5 μM | Mp 131-132 Orange solid |
| 28 | 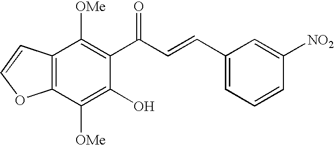 | 369 | 5-10% block at 5 μM | Mp 97-98 Dark brown crystals |
| 29 | 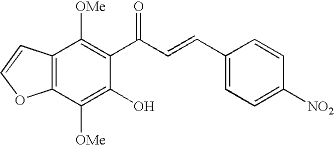 | 369 | Stocks precipitate | Mp 148 Brown needles |
| 30 | 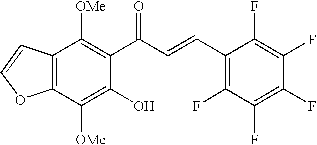 | 414 | K_{d peak} = 5 μM K_{d end} = 3.5 μM | Mp 112-114 Orange solid |
| 31 | 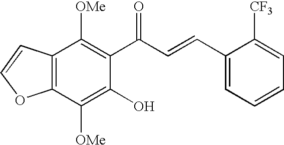 | 392 | K_{d peak} = 40 μM block not phasic | Mp 150-151 Orange granules |
| 32 | 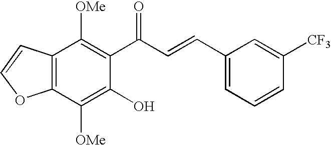 | 392 | K_{d peak} = 40 μM block not phasic | Mp 123 Orange needles |
| 33 | 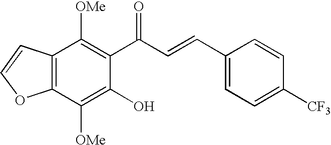 | 392 | K_{d peak} = 10 μM K_{d end} = 4 μM | Mp 135 Red-brown needles |
| 34 | 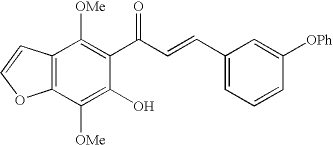 | 416 | no effect at 5 μM Not tested against other channels | Mp 111 Orange needles |
| 35 | 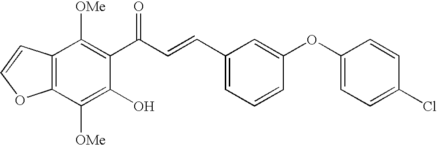 | 450.5 | no effect at 5 μM Not tested against other channels | Mp 107-108 Orange needles |
| 36 | 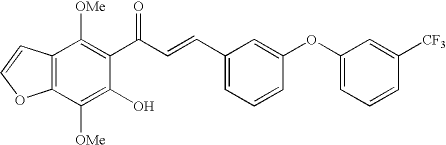 | 484 | no effect at 5 μM Not tested against other channels | Mp 121 Orange granular crystals |
| 37 | 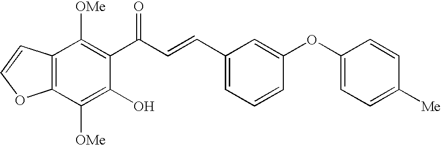 | 430 | no effect at 1 μM Not tested against other channels | Mp 126 Red prisms |
| 38 | 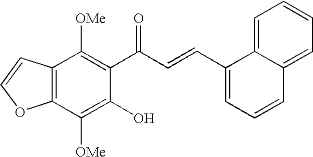 | 374 | no effect at 5 μM Not tested against other channels | Mp 131 Orange prisms |
| 39 | 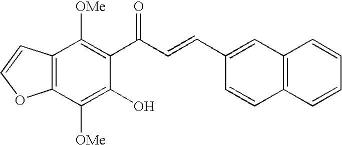 | 374 | no effect at 5 μM Not tested against other channels | Mp 158 Orange granular crystals |
| 40 | 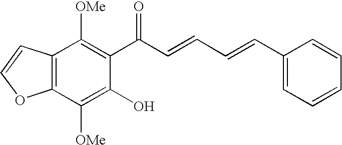 | 349 | no effect at 1 μM Not tested against other channels | Mp 112 pale brown granular crystals |
| 41 | 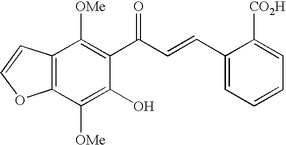 | 368 | no effect at 20 μM Not tested against other channels | Mp 131 Orange yellow granules |
| 42 | 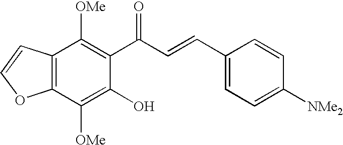 | 367 | K_{d peak} = 14 μM K_{d end} = 5 μM | Mp 61 Red-brown prisms |
| 43 | 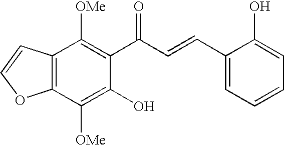 | 340 | K_{d peak} = 16 μM K_{d end} = 8 μM | Mp 98 Mustard yellow granular crystals |
| 44 | 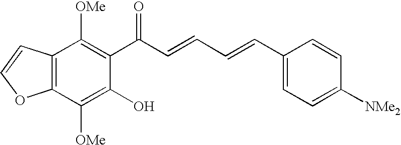 | 393 | K_{d peak} = 22 μM Block not phasic | Mp 99 Red-brown prisms |
| 45 | 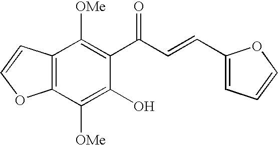 | 314 | K_{d peak} = 20 μM K_{d end} = 4 μM | Mp 127 Orange granules |
| 46 | 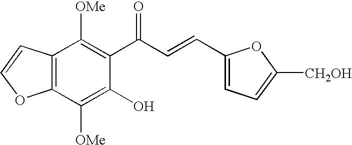 | 344 | no effect at 20 μM Not tested against other channels | Mp 120-121 Orange granules |
| 47 | 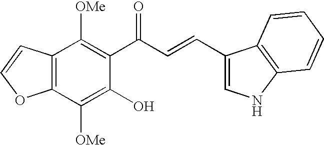 | 363 | no effect at 5 μM Not tested against other channels | Mp 138-140 Orange prisms |
| 48 | 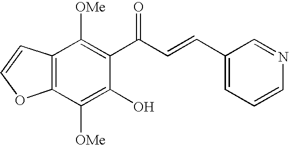 | 325 | K_{d peak} = 20 μM K_{d end} = 9 μM | Mp 110 Orange granules |
| 49 | 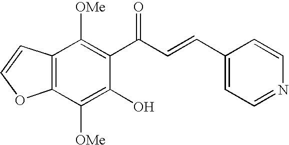 | 325 | K_{d peak} = 20 μM block not phasic | Brown amorphous resin |
| 50 | 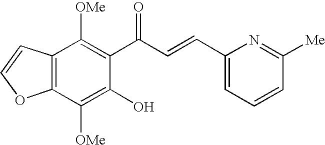 | 339 | K_{d peak} = 12 μM block not phasic | Mp 86-87 Pale brown granules |
| 51 | 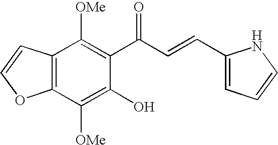 | 313 | K_{d peak} = 30 μM K_{d end} = 9 μM | Mp 93 Dark brown prisms |
| 52 | 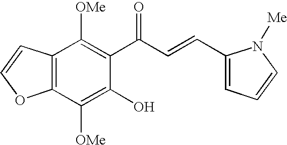 | 327 | K_{d peak} = 40 μM K_{d end} = 15 μM | Mp 87-88 Dark brown prisms |
| 53 | 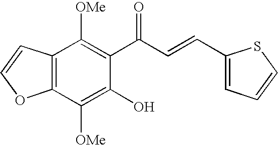 | 330 | K_{d peak} = 1.75 μM K_{d end} = 300 nM | Cytotoxic Mp 129 Red needles |
| 54 | 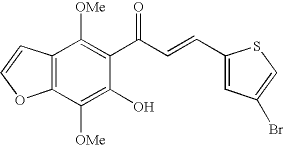 | 409 | K_{d peak} = 4 μM K_{d end} = 250 nM | Cytotoxic Mp 139 Brown needles |
| 55 | 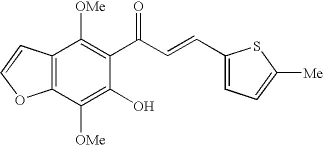 | 344 | K_{d peak} = 3 μM K_{d end} = 500 nM | Cytotoxic Mp 103 Orange prisms |
| 56 | 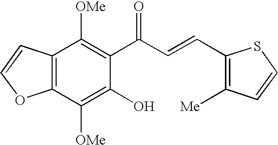 | 344 | K_{d peak} = 25 μM K_{d end} = 6 μM | Mp 131 Brown needles |
| 57 | 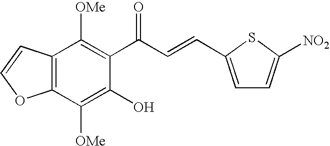 | 375 | No effect at 10 μM | Mp 127 Dark brown granules |
| 58 | 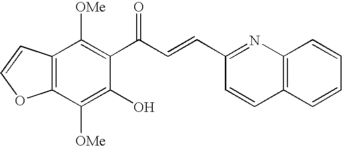 | 375 | No effect at 5 μM | Mp 150 Brown granules |
| 59 | 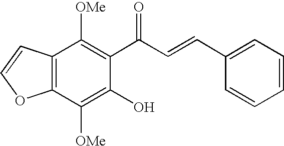 | 338 | K_{d peak} = 40 μM K_{d end} = 5 μM | Amorphous resin |
| 60 | 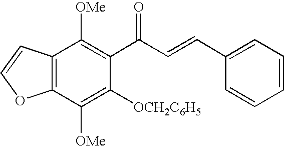 | 414 | K_{d peak} = 10 μM K_{d end} = 200 nM | |
| 61 | 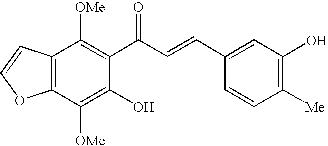 | 354 | | |
| 62 | 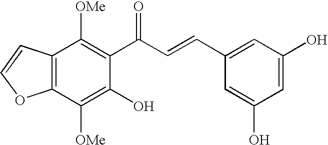 | 356 | | |
| 63 | 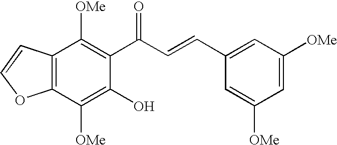 | 384 | | |
| 64 | 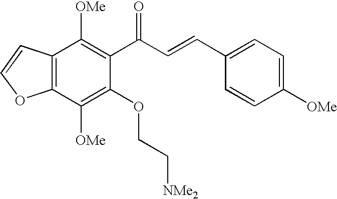 | 425 | | |
| 65 | 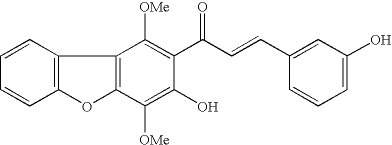 | 392 | | |
| 66 | 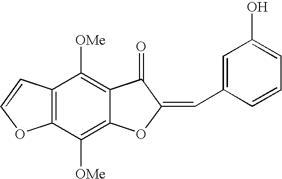 | 338 | | |
| 67 | 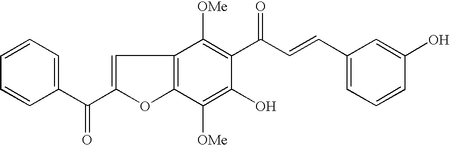 | 444 | | |
| 68 | 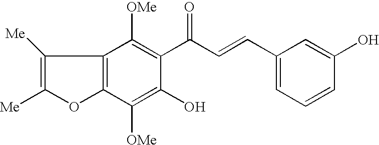 | 368 | | |
| 69 | 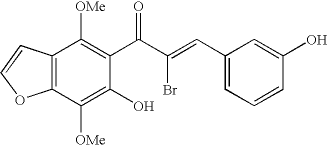 | 419 | | |
| 70 | 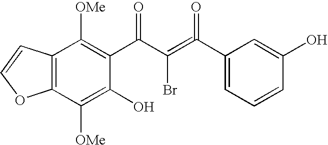 | 435 | | |
| 71 | 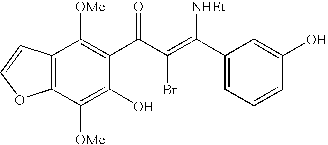 | 462 | | |
| 72 | 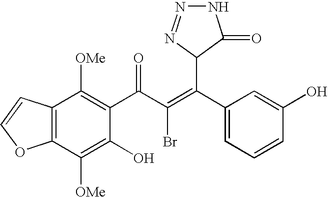 | 502 | | |
| 73 | 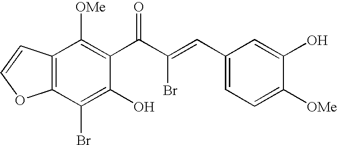 | 498 | | |
| 74 | 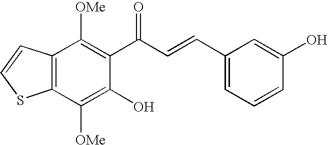 | 356 | | |
| 75 | 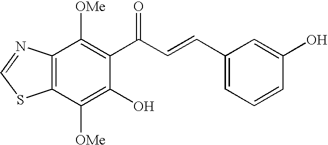 | 357 | | |
| 76 | 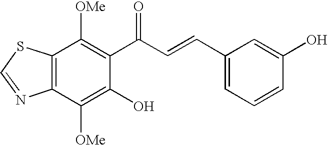 | 357 | | |
| 77 | 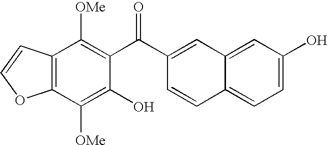 | 364 | | |
| 78 | 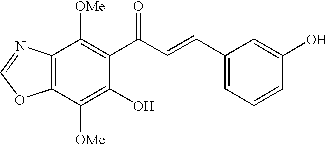 | 341 | | |
| 79 | 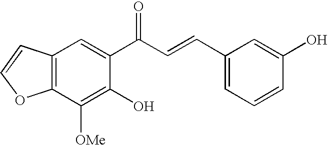 | 310 | | |
| 80 | 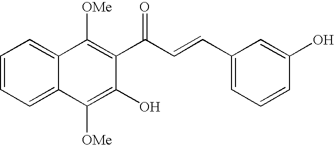 | 350 | | |
| 81 | 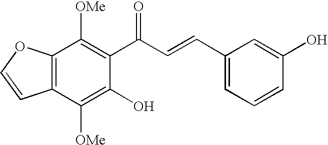 | 340 | | |
| 82 | 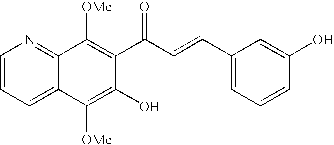 | 351 | | |
| 83 | 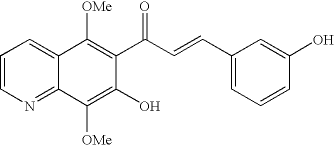 | 351 | | |
| 84 | 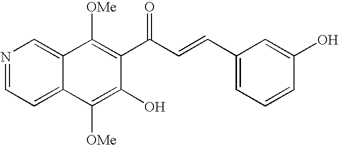 | 351 | | |
| 85 | 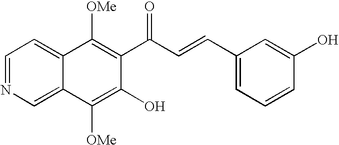 | 351 | | |
| 86 | 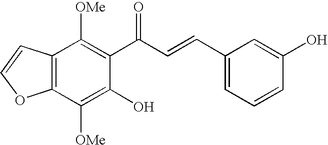 | 352 | | |
| 87 | 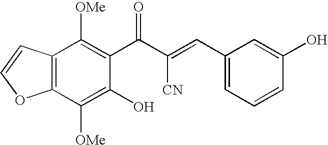 | 365 | | |
| 88 | 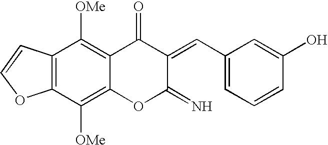 | 365 | | |
| 89 | 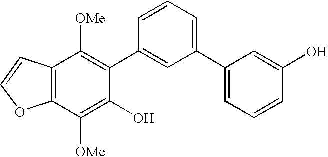 | 362 | | |
| 90 | 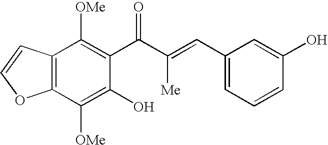 | 354 | | |
| 91 | 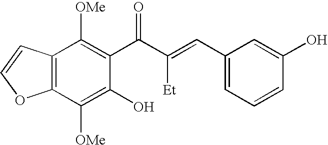 | 368 | | |
| 92 | 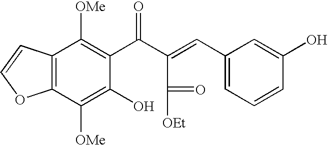 | 412 | | |
| 93 | 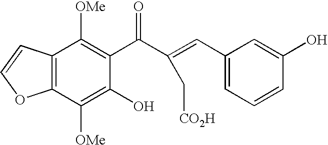 | 398 | | |
| 94 | 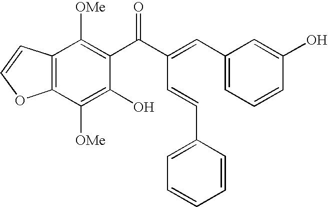 | 442 | | |
| 95 | 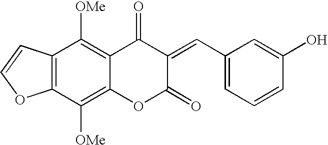 | 366 | | |
| 96 | 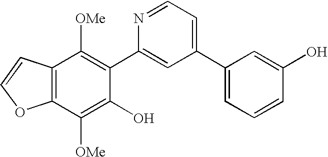 | 363 | | |
| 97 | 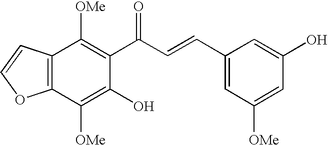 | 370 | | |
| 98 | 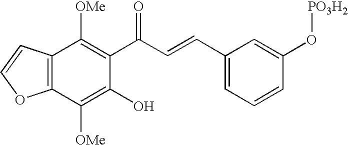 | 419 | | |
| 99 | 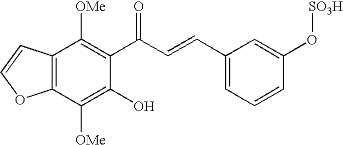 | 420 | | |
| 100 | 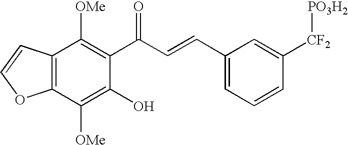 | 452 | | |
| 101 | 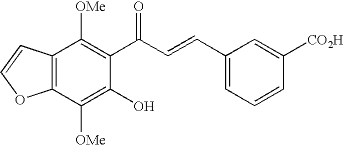 | 368 | | |
| 102 | 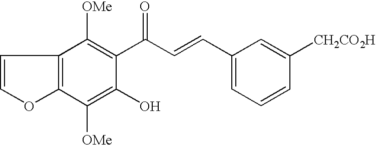 | 382 | | |
| 103 | 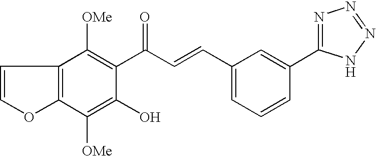 | 392 | | |
| 104 | 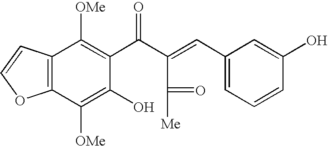 | 382 | | |
| 105 | 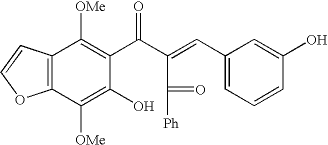 | 444 | | |
| 106 | 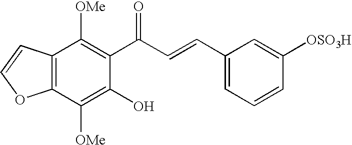 | 420 | | |
| 107 | 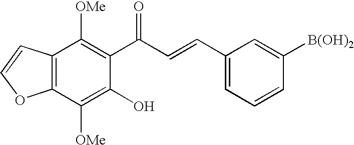 | 368 | | |
| 108 | 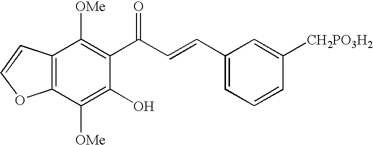 | 418 | | |
| 109 | 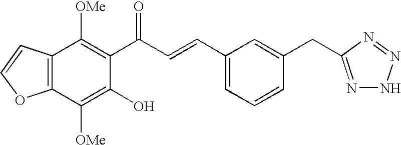 | 406 | | |
| 110 | 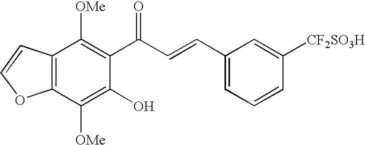 | 453 | | |
| 111 | 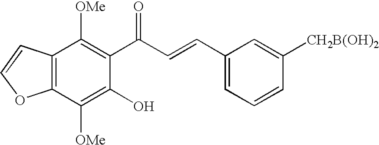 | 382 | | |
| 112 | 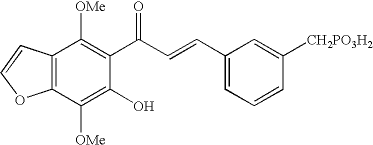 | 430 | | |
| 113 | 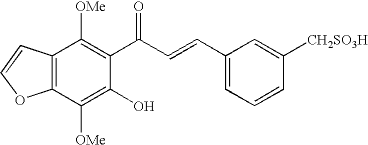 | 433 | | |
| 114 | 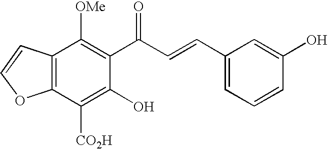 | | | |

Proliferation Test
[³H]-Thymidine Incorporation Assay

Resting peripheral blood mononuclear cells from healthy volunteers were seeded at 2×10⁵ cells per well in medium (RPMI 1640 supplemented 10% fetal calf serum, 2 mM glutamine, 1 mM sodium pyruvate, 1% nonessential amino acids, 100 units/ml penicillin, 100 μg/ml streptomycin and 50 μM β-mercaptoethanol) in flat-bottom 96 well plates (final volume 200 μl). Cells pre-incubated with drug (60 min), were stimulated with 5 ng/ml anti-CD3 Ab) for 48 h. [³H]-Thymidine (1 μCi per well) was added for the last 6 h. Cells were harvested onto glass fibre filters and radioactivity measured in a scintillation counter. All experiments were done in triplicate. Results are reported as normalised for maximum [³H]-thymidine incorporation for controls.

Proliferation Restults

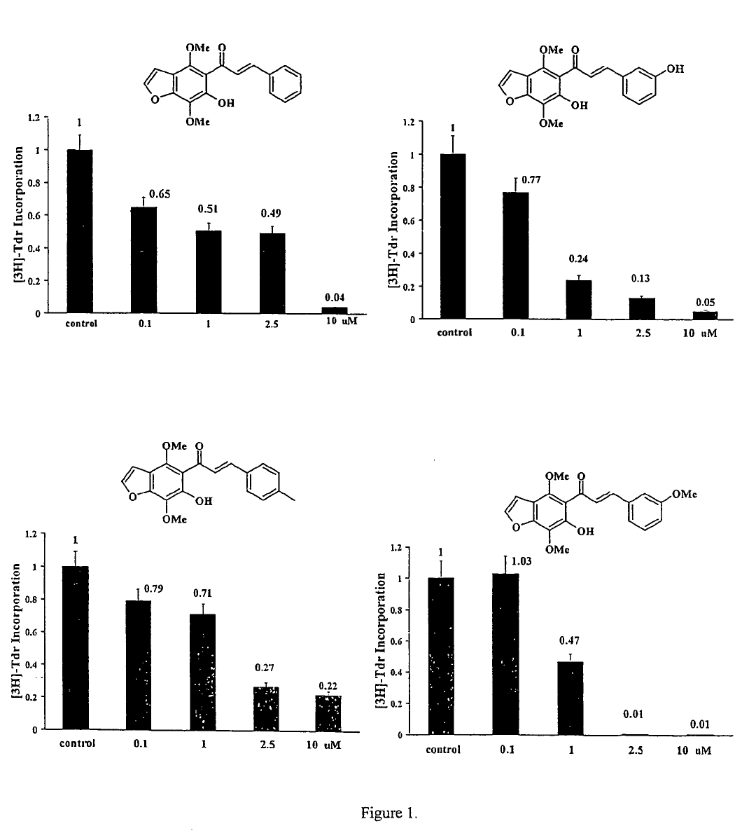

The proliferation results for Example 1 and 18 are shown in FIG. 1. As will been seen from these results, the compound of Example 1 suppresses proliferation of human peripheral blood lymphocytes with an EC50 of 1 μM, Example 18 with an EC50 of 500 nM, Example 23 with an EC50 of 1.5 μM and Example 24 with an EC50 of 1 μM.

Flow Cytometric Measurement of Cell Viability

Jurkat E6-1 and MEL were seeded at 5×10⁵ cells/ml in twelve-well plates. Drug (100 nM, 1 μM, 2.5 μM and 10 μM) was added in a final DMSO concentration of 0.1%. After 48 h of incubation, cells were harvested by sucking them off the plates. Cells were centrifuged, resuspended in 0.5 ml PBS containing 1 μg/ml propidium iodide (PI), and red fluorescence measured on a FACScan flow cytometer (Becton Dickinson) after 20 min (10⁴ cells of every sample being analyzed). The percentage of dead cells was determined by their PI uptake. Incubation with 20% DMSO served as a control for setting the gates of the flow cytometer for dead cells. The results are shown in Table 2.

TABLE 2

| Compounds | MEL cells | Jurkat T-cells |
|---|---|---|
| Control 1 | 3.06% | 2.67% |
| (O.1% DMSO) | | |
| Control 2 | 99.10% | 97.90% |
| (20% DMSO) | | |
| Example 1 | 4.95% | 3.02% |
| 100 nM | | |
| Example 1 | 6.21% | 1.47% |
| 1 μM | | |
| Example 1 | 6.70% | 1.78% |
| 2.5 μM | | |
| Example 1 | 5.88% | 8.10% |
| 10 μM | | |
| Example 18 | 6.89% | 2.57% |
| 100 nM | | |
| Example 18 | 3.60% | 2.22% |
| 1 μM | | |
| Example 18 | 6.98% | 2.59% |
| 2.5 μM | | |
| Example 18 | 4.41% | 4.70% |
| 10 μM | | |
| Example 24 | 3.53% | 2.41% |
| 100 nM | | |
| Example 24 | 3.73% | 2.81% |
| 1 μM | | |
| Example 24 | 5.26% | 2.31% |
| 2.5 μM | | |
| Example 24 | 3.00% | 9.8% |
| 10 μM | | |

From the above results it is apparent that the compound of Example 1 has significant therapeutic potential. It blocks the Kv1.3 voltage gated potassium channel in T-lymphocytes, with a Kd (dissociation constant) of 400 nM. Thus, in blocking the Kv1.3 channel in T-lymphocytes, this compound inhibit the immune response, as measured below by the inhibition of T-lymphocyte proliferation in response to stimulation by anti-CD3 antibody (FIG. 1). Furthermore, example 1 is non-cytotoxic in-vitro (Table 2) and non-toxic when 30 uM is injected intravenously into mice.

Further preferred examples of compounds of the invention include Examples 18 and 24. These compounds have been found to also be non-cytotoxic (see Table 2), non-toxic when injected intravenously into mice, and even more potently antiproliferative (FIG. 1).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference to any prior art in this specification is not, and should not be taken as an acknowledgment or any form or suggestion that that prior art forms part of the common general knowledge in Australia.

It would be appreciated by a person skilled in the art the numerous variations and/or modifications may be made to the invention as shown the specific embodiments without departing from the spirit or scope of the invention as broadly described. The present embodiments are, therefore, to be considered in all respects as illustrative and not restrictive.

1. A method of intentionally modulating potassium ion channel activity of T-cells by the administration of an effective amount of a compound of Formula I 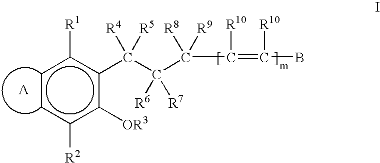
wherein ring A is an optionally substituted fused carbocyclic or heterocyclic ring;
B is an optionally substituted aromatic or heteroaromatic ring;
R¹ and R² are independently selected from hydrogen, cyano, halo, nitro, optionally substituted alkyl, optionally substituted alkenyl, optionally substituted alkynyl, optionally substituted cycloalkyl —OR, —C(O)R, —C(O)OR, —OC(O)R (where R is hydrogen or is selected from an optionally substituted alkyl, alkenyl, alkynyl, cycloalkyl and aryl group), —C(O)NR′R″, —NR′C(O)R″ and —NR′R″ (where R′ and R″ are independently selected from hydrogen and lower alkyl);
R³ is hydrogen or optionally substituted alkyl, alkenyl or alkynyl group;
R⁴ and R⁵ are independently selected from hydrogen, hydroxy, alkyl, alkenyl; alkynyl and alkoxy;
or R⁴ and R⁵ together are ═O, ═S, ═NR or ═NOR, (where R is hydrogen or lower alkyl);
R⁶ and R⁷ are independently selected from hydrogen, cyano, halo, nitro, optionally substituted alkyl, optionally substituted alkenyl, optionally substituted alkynyl optionally substituted cycloalkyl, —OR, —C(O)R, —C(O)OR, —OC(O)R (where R is hydrogen or is selected from an optionally substituted alkyl, alkenyl, alkynyl, cycloalkyl and aryl group), —C(O)NR′R″ and —NR′R″ (where R′ and R″ are independently selected from hydrogen and lower alkyl);
or R³ together with R⁷ together with the atoms to which they are attached form an optionally substituted five or six membered heterocyclic ring;
R⁸ and R⁹ are independently selected from hydrogen, cyano, halo, nitro, a 5- or 6-membered nitrogen containing heterocyclic ring, optionally substituted alkyl, optionally substituted alkenyl, optionally substituted alkynyl, optionally substituted cycloalkyl, optionally substituted arylalkyl, optionally substituted heterocyclylalkyl, —OR, —C(O)R, —C(O)OR, —OC(O)R (where R is hydrogen or is selected from an optionally substituted alkyl, alkenyl, alkynyl, cycloalkyl or aryl group), —C(O)NR′R″, —NR′C(O)R″ and —NR′R″ (where R′ and R″ are independently selected from hydrogen and lower alkyl);
or R⁸ and R⁹ are together ═O, ═S, ═NR or ═NOR, (where R is hydrogen or lower alkyl);
or R⁶ and R⁸ together form a bond;
or R⁴, R⁵, R⁶, R⁸ and R⁹ together with the atoms to which they are attached form an aromatic or heteroaromatic ring;
or R⁶, R⁷ and R⁸ and the atoms to which they are attached, together with a ring atom of B form a six membered aromatic or heteroaromatic ring fused to ring B;
m=0, 1 or 2;
each R¹⁰ is independently selected from hydrogen, cyano, halo, nitro, optionally substituted alkyl, optionally substituted alkenyl, optionally substituted alkynyl and optionally substituted cycloalkyl;
with the proviso that R³ is not —CH₂CO₂H when R¹ and R² are methoxy, m is 0, R⁴ and R⁵ together are ═O, R⁶ and R⁸ together form a bond, R⁷ and R⁹ are hydrogen, ring A is an unsubstituted furyl ring and B is an optional substituted phenyl ring;
and with the proviso that when R¹ and R² are methoxy, R³ is hydrogen, m is 0, R⁴ and R⁵ together are ═O, B is an optional substituted phenyl ring and one of R⁸ or R⁹ is hydrogen the other of R⁸ or R⁹ is not —CH₂CN or optionally substituted forms thereof;
and with the proviso that ring A is not an unsubstituted cyclopentadiene ring, when R¹ and R² are methoxy, R³ is hydrogen, R⁴ and R⁵ together are ═O, R⁶ and R⁸ together form a bond, R⁷ and R⁹ are hydrogen and B is an optionally substituted phenyl or pyridine ring;
and with the proviso that that R³ is not —(CH₂)₂NR′R″ (where R′ and R″ are independently hydrogen or alkyl, or together with the nitrogen to which they are attached form an unsubstituted piperidine ring), when R¹ and R² are methoxy, R⁴ is hydroxy, R⁵, R⁶, I R⁸ and R⁹ are hydrogen, ring A is a five membered heterocyclic ring containing oxygen, and B is an optionally substituted phenyl ring;
or its salt or pharmaceutically acceptable derivative thereof.

2. A method for the treatment or prevention of autoimmune or chronic inflammatory diseases, or the prevention of rejection of foreign organ transplants and/or related afflictions, by the administration to a patient in need of treatment of an effective amount of a compound of Formula I 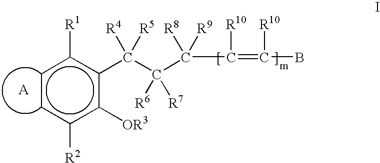
wherein ring A is an optionally substituted fused carbocyclic or heterocyclic ring;
B is an optionally substituted aromatic or heteroaromatic ring;
R¹ and R² are independently selected from hydrogen, cyano, halo, nitro, optionally substituted alkyl, optionally substituted alkenyl, optionally substituted alkynyl, optionally substituted cycloalkyl, —OR, —C(O)R, —C(O)OR, —OC(O)R (where R is hydrogen or is selected from an optionally substituted alkyl, alkenyl, alkynyl, cycloalkyl and aryl group), —C(O)NR′R″, —NR′C(O)R″ and —NR′R″ (where R′ and R″ are independently selected from hydrogen and lower alkyl);
R³ is hydrogen or optionally substituted alkyl, alkenyl or alkynyl group;
R⁴ and R⁵ are independently selected from hydrogen, hydroxy, alkyl, alkenyl; alkynyl and alkoxy;
or R⁴ and R⁵ together are ═O, ═S, ═NR or ═NOR, (where R is hydrogen or lower alkyl);
R⁶ and R⁷ are independently selected from hydrogen, cyano, halo, nitro, optionally substituted alkyl, optionally substituted alkenyl, optionally substituted alkynyl optionally substituted cycloalkyl, —OR, —C(O)R, —C(O)OR, —OC(O)R (where R is hydrogen or is selected from an optionally substituted alkyl, alkenyl, alkynyl, cycloalkyl and aryl group), —C(O)NR′R″ and —NR′R″ (where R′ and R″ are independently selected from hydrogen and lower alkyl);
or R³ together with R⁷ together with the atoms to which they are attached form an optionally substituted five or six membered heterocyclic ring;
R⁸ and R⁹ are independently selected from hydrogen, cyano, halo, nitro, a 5- or 6-membered nitrogen containing heterocyclic ring, optionally substituted alkyl, optionally substituted alkenyl, optionally substituted alkynyl, optionally substituted cycloalkyl, optionally substituted arylalkyl, optionally substituted heterocyclylalkyl, —OR, —C(O)R, —C(O)OR, —OC(O)R (where R is hydrogen or is selected from an optionally substituted alkyl, alkenyl, alkynyl, cycloalkyl or aryl group), —C(O)NR′R″, —NR′C(O)R″ and —NR′R″ (where R′ and R″ are independently selected from hydrogen and lower alkyl);
or R⁸ and R⁹ are together ═O, ═S, ═NR or ═NOR, (where R is hydrogen or lower alkyl);
or R⁶ and R⁸ together form a bond;
or R⁴, R⁵, R⁶, R⁸ and R⁹ together with the atoms to which they are attached form an aromatic or heteroaromatic ring;
or R⁶, R⁷ and R⁸ and the atoms to which they are attached, together with a ring atom of B form a six membered aromatic or heteroaromatic ring fused to ring B;
m=0, 1 or 2;
each R¹⁰ is independently selected from hydrogen, cyano, halo, nitro, optionally substituted alkyl, optionally substituted alkenyl, optionally substituted alkynyl and optionally substituted cycloalkyl;
with the proviso that R³ is not —CH₂CO₂H when R¹ and R² are methoxy, m is 0, R⁴ and R⁵ together are ═O, R⁶ and R⁸ together form a bond, R⁷ and R⁹ are hydrogen, ring A is an unsubstituted furyl ring and B is an optional substituted phenyl ring;
and with the proviso that when R¹ and R² are methoxy, R³ is hydrogen, m is 0, R⁴ and R⁵ together are ═O, B is an optional substituted phenyl ring and one of R⁸ or R⁹ is hydrogen the other of R⁸ or R⁹ is not —CH₂CN or optionally substituted forms thereof;
and with the proviso that ring A is not an unsubstituted cyclopentadiene ring, when R¹ and R² are methoxy, R³ is hydrogen, R⁴ and R⁵ together ar ═O, R⁶ and R⁸ together form a bond, R⁷ and R⁹ are hydrogen and B is an optionally substituted phenyl or pyridinyl ring;
and with the proviso that that R³ is not —(CH₂)₂N′R″ (where R′ and R″ are independently hydrogen or alkyl, or together with the nitrogen to which they are attached form an unsubstituted piperidine ring), when R¹ and R² are methoxy, R⁴ is hydroxy, R⁵, R⁶, R⁷, R⁸ and R⁹ are hydrogen, ring A is a five membered heterocyclic ring containing oxygen, and B is an optionally substituted phenyl ring;
or pharmaceutically acceptable derivative thereof.

3. The method of claim 1 or 2 wherein the fused ring A is an optionally substituted ring selected from the following 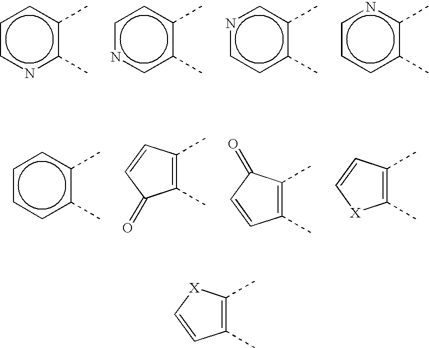
where X is O, S or NR, where R is hydrogen, lower alkyl or oxygen; 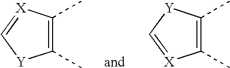
where X is N, and Y is O, S or NR and R is hydrogen, lower alkyl or oxygen;
and where the two dashed lines on the right hand side of the rings indicate the location at which the ring A is fused to the phenyl ring.

4. The method of claim 3 wherein the fused ring A is an optionally substituted ring selected from the following:— 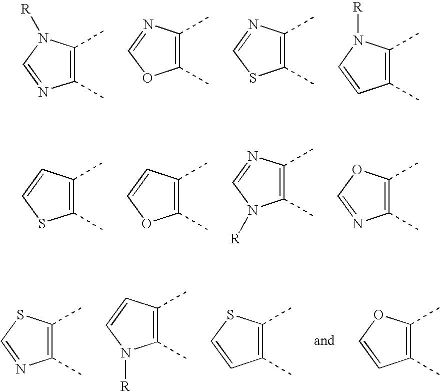
where R is hydrogen or lower alkyl.

5. The method of claim 3 or 4 wherein the fused ring A is optionally substituted with halo, lower alkyl, benzyl or —C(O)C₆H₅;
R¹ and R² are independently selected from hydrogen; halogen; hydroxy; lower alkoxy, optionally substituted benzyl, optionally substituted phenyl, optionally substituted diphenyl, optionally substituted phenoxy and optionally substituted benzoxy group;
R³ is hydrogen, methyl or benzyl optionally substituted with 1 to 3 halo or lower alkyl groups;
R⁴ and R⁵ are independently hydrogen or hydroxy, or together are ═O;
R⁶ is selected from hydrogen, halogen, —CN, —C(O)R (where R is lower alkyl or phenyl), —C(O)OR, (where R is hydrogen or lower alkyl), optionally substituted alkyl, and optionally substituted alkenyl group;
R⁷ is hydrogen;
R⁸ and R⁹ are independently selected from hydrogen; lower alkyl, —CHR(CN) (where R is selected from hydrogen, OH, lower alkyl and lower alkoxy), —C(O)R (where R is optionally substituted lower alkyl, optionally substituted lower alkoxy or optionally substituted phenyl), —NR′R″ (where R′ and R″ are independently selected from hydrogen or lower alkyl), and 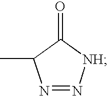
or R⁶ an R⁸ together form a bond between the carbons to which they are attached;
m is 0 or 1.

6. The method of any one of claims 3 to 5 wherein B is an optionally substituted ring selected from phenyl, naphthalenyl, pyrrolyl, furyl, thiophenyl, imidazolyl, pyrazolyl, thiazolyl, oxazolyl, pyridinyl, pyryl, pyrimidinyl, indolyl, quinolinyl, isoquinolinyl and a ring system of the structure C 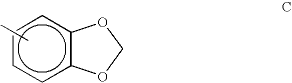
and the ring B is optionally substituted with one or more substituents independently selected from
a) halo, cyano, —NO₂, —SO₃, —OSO₃H, —OPO₃H₂, —PO₃ and —B(OH)₂;
b) —NR′R″ (where R′ and R″ are independently hydrogen or lower alkyl);
c) —NR′C(O)R″ (where R′ and R″ are independently hydrogen or lower alkyl);
d) phenyl and tetrazolyl;
e), —OR, —C(O)R, and —C(O)OR (where R is hydrogen, optionally substituted lower alkyl, optionally substituted phenyl, optionally substituted phenylloweralkyl and the optional substituents are independently selected from lower alkyl, halo and —NR′R″ where R′ and R″ are independently hydrogen or lower alkyl);
f —C(O)NHSO₂R′″ and —S(O)₂NHC(O)R′″ (where R′″ is lower alkyl);
g) optionally substituted lower alkyl such as —CH₃, —CH(CH₃)₂, —CH₂B(OH)₂, —CH₂PO₃, —CH₂SO₃, —CH₂OPO₃H₂, —CH₂OSO₃H, —CH₂C(O)NHSO₂R′″, —CH₂S(O)₂NHC(O)R′″ (where R′″ is lower alkyl), —CH₂C₆H₅, —CH₂-tetrazolyl, —(CH₂)ₙNR′R″ (where n is from 1 to 4 and R′ and R″ are independently hydrogen or lower alkyl), —CF₃, —CF₂B(OH)₂, —CF₂PO₃, —CF₂SO₃, —CF₂OPO₃H₂, —CF₂OSO₃H, —CF₂C(O)NHSO₂R′″, —CF₂S(O)₂NHC(O)R′″ where R′″ is lower alkyl, —CF₂C₆H₅ and —CF₂-tetrazolyl.

7. The method of claim 6 wherein ring B is substituted by —(CH₂)ₙR²⁰ where n is from 0 to 6 and R²⁰ is selected from —OSO₃H, —OPO₃H₂, —CO₂H, tetrazolyl, —B(OH)₂, —S(O)₂NHC(O)R′, —C(O)NHS(O)₂R′ (where R′ is lower alkyl), —OH, —C₆H₄OH, —CF₂PO₃ and —SO₃.

8. The method of claim 6 or 7 comprising the administration of a compound of the formula II or a pharmaceutically acceptable derivative thereof 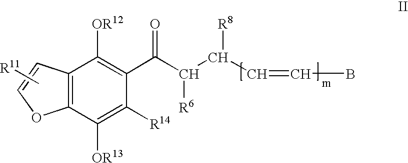
where R⁶ and R⁸ are hydrogen or together form a double bond, and R¹¹ is hydrogen, lower alkyl, halogen or —C(O)C₆H₅, R¹² and R¹³ are independently selected from hydrogen, alkyl, optionally substituted phenyl, optionally substituted benzyl, —(CH₂)ₙNR′R″ (where n is from 1 to 4 and R′ and R″ are independently hydrogen or lower alkyl) and —(CH₂)ₙR²⁰, where n is from 1 to 4, and R²⁰ is selected from —OSO₃H, —OPO₃H₂, —CO₂H, tetrazolyl, —B(OH)₂, —S(O)₂NHC(O)R and —C(O)NHS(O)₂R (where R is lower alkyl), R¹⁴ is hydroxy or alkoxy; m is 0 or 1 and B is as defined in claim 6 or 7.

9. The method of claim 8 comprising the administration of a compound of the formula III or a pharmaceutically acceptable derivative thereof 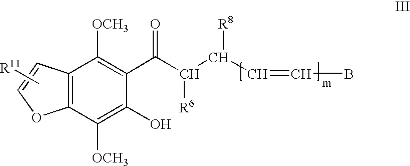
where R⁶, R⁸, R¹¹, m and B are as defined in claim 8.

10. The method of claim 9 comprising the administration of a compound of the formula IV or a pharmaceutically acceptable derivative thereof 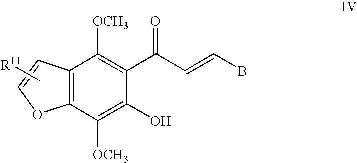
where R¹¹ is as defined in claim 9 and B is an optionally substituted ring or ring system selected from phenyl, naphthalenyl pyridinyl, pyrrolyl, furyl, indolyl, quinolinyl, isoquinolinyl, thiophenyl, 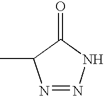
and

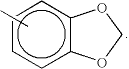

11. The method of claim 10 wherein B is optionally substituted with one or more substituents independently selected from —OPO₃H₂, —PO₃, —OSO₃, —SO₃, —CH₂PO₃, —CH₂SO₃, —CO₂H, —CH₂CO₂H, —CF₂PO₃, —CF₂SO₃, —OH, —B(OH)₂, —OCH₃, —OCH₂CH₃, —CF₃, —CH₃, —CH₂CH₃, —CH(CH₃)₂, —C₆H₅, —OC₆H₅—OC₆H₄CH₃, -tetrazolyl, —CH₂tetrazolyl, —CF₂tetrazolyl, —NHC(O)CH₃, —F, —Cl, —Br, —CN, —OCH₂N(CH₂CH₃)₂, —NO₂, —N(CH₃)₂ and

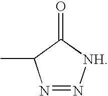

12. The method of claim 1 or 2 comprising the administration of a compound of the formula V or a pharmaceutically acceptable derivative thereof 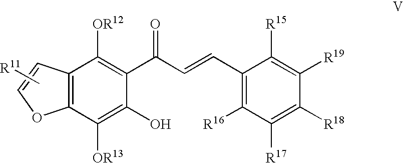
wherein R¹¹ is hydrogen, lower alkyl, halogen or —C(O)C₆H₅; R¹² and R¹³ are independently selected from hydrogen, alkyl, optionally substituted phenyl and optionally substituted benzyl;
R¹³ is also selected from —(CH₂)ₙNR′R″ (wherein is from 1 to 4 and R′ and R″ are independently hydrogen or lower alkyl) and —(CH₂)ₙR²⁰ (where n is from 0 to 6 and R²⁰ is selected from —OSO₃H, —OPO₃H₂, —CO₂H, -tetrazolyl, —B(OH)₂, —S(O)₂NHC(O)R and —C(O)NHS(O)₂R ere R is lower alkyl);
R¹⁵, R¹⁶, R¹⁷ and R¹⁸ are independently selected from hydrogen, —OPO₃H₂, —PO₃, —OSO₃, —SO₃, —CH₂PO₃, —CH₂SO₃, —CO₂H, —CH₂CO₂H, —CF₂PO₃, —CF₂SO₃, —OH, —B(OH)₂, —OCH₃, —OCH₂CH₃, CF₃, —CH₃, —CH₂CH₃, —CH(CH₃)₂, —C₆H₅, —OC₆H₅—OC₆H₄CH₃, -tetrazolyl, —CH₂tetrazolyl, —CF₂tetrazolyl, —NHC(O)CH₃, —F, —Cl, —Br, —CN, —OCH₂CH₂N(CH₂CH₃)₂, —NO₂, —N(CH₃) and 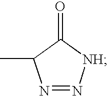
R¹⁹ is selected from —(CH₂)ₙR²⁰, where n is from 0 to 6, and R²⁰ is selected from hydrogen (when n is other than 0), —OSO₃H, —OPO₃H₂, —CO₂H, -tetrazolyl, —B(OH)₂, ⁻S(O)₂NHC(O)R′, —C(O)NHS(O)₂R′, —OR (where R′ is lower alkyl), —OR—C₆H₄OH, —CF₂PO₃ and —SO₃.

13. The method of claim 1 or 2 comprising the administration of a compound of the formula VI 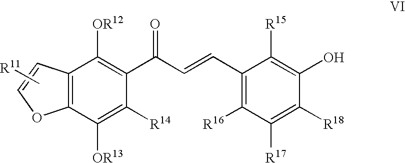
wherein R¹¹ is hydrogen, lower alkyl, halogen or —C(O)C₆H₅;
R¹² and R¹³ are independently selected from hydrogen, alkyl, optionally substituted phenyl and optionally substituted benzyl;
and R¹³ is also selected from —(CH₂)ₙNR′R″ (where n is from 1 to 4 and R′ and R″ are independently hydrogen or lower alkyl) and —(CH₂)ₙR²⁰, (where n is from 0 to 6, and R²⁰ is selected from —OSO₃H, —OPO₃H₂, —CO₂H, tetrazolyl, —B(OH)₂, —S(O)₂NHC(O)R and —C(O)NHS(O)₂R where R is lower alkyl);
R¹⁴ is hydroxy, alkoxy, —(CH₂)ₙNR′R″ (where n is from 1 to 4 and R′ and R″ are independently hydrogen or lower alkyl) or —(CH₂)ₙR²⁰, (where R²⁰ is selected from —OSO₃H, —OPO₃H₂, —CO₂H, tetrazolyl, —B(OH)₂, —S(O)₂NHC(O)R and —S(O)₂NHS(O)₂R where R is lower alkyl;
R¹⁵, R¹⁶, R¹⁷ and R¹⁸ are independently selected from hydrogen, —OPO₃H₂, —PO₃, —OSO₃, —SO₃, —CH₂PO₃, —CH₂SO₃, CO₂H, —CH₂CO₂H, —CF₂PO₃, —CF₂SO₃, —OH, —B(OH)₂, —OCH₃, —OCH₂CH₃, —CF₃, —CH₃, —CH₂C)H, —CH(CH₃)₂, —C₆H₅, —OC₆H₅—OC₆H₄CH₃, -tetrazolyl, —CH₂tetrazolyl, —CF₂tetrazolyl, —NHC(O)CH₃, —F, —Cl, —Br, —CN, —OCH₂CH₂N(CH₂CH₃)₂, —NO₂, —N(CH₃) and

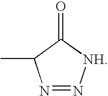

14. The method of any one of claims 8 to 13 wherein R¹¹ is hydrogen.

15. A compound of formula I 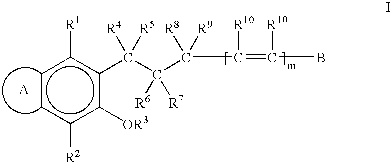
wherein ring A is an optionally substituted sed carbocyclic or heterocyclic ring;
B is an optionally substituted aromatic or heteroaromatic ring;
R¹ and R² are independently selected from hydrogen, cyano, halo, nitro, optionally substituted alkyl, optionally substituted alkenyl, optionally substituted alkynyl, optionally substituted cycloalkyl, —OR, —C(O)R, —C(O)OR, —OC(O)R (where R is hydrogen or is selected from an optionally substituted alkyl, alkenyl, alkynyl, cycloalkyl and aryl group), —C(O)NR′R″, —NR′C(O)R″ and —NR′R″ (where R′ and R″ are independently selected from hydrogen and lower alkyl);
R³ is hydro n or optionally substituted alkyl, alkenyl or alkynyl group;
R⁴ and R⁵ are independently selected from hydrogen, hydroxy, alkyl, alkenyl; alkynyl and alkoxy;
or R⁴ and R⁵ together are ═O, ═S, ═NR or ═NOR, (where R is hydrogen or lower alkyl);
R⁶ and R⁷ are independently selected from hydrogen, cyano, halo, nitro, optionally substituted alkyl, optionally substituted alkenyl, optionally substituted alkynyl optionally substituted cycloalkyl, —OR, —C(O)R, —C(O)OR, —OC(O)R (where R is hydrogen or is selected from an optionally substituted alkyl, alkenyl, alkynyl, cycloalkyl and aryl group), —C(O)NR′R″ and —NR′R″ (where R′ and R″ are independently selected from hydrogen and lower alkyl);
or R³ together with R⁷ together with the atoms to which they are attached form an optionally substituted five or six membered heterocyclic ring;
R⁸ and R⁹ are independently selected from hydrogen, cyano, halo, nitro, a 5- or 6-membered nitrogen containing heterocyclic ring, optionally substituted alkyl, optionally substituted alkenyl, optionally substituted alkynyl, optionally substituted cycloalkyl, optionally substituted arylalkyl, optionally substituted heterocyclylalkyl, —OR, —C(O)R, —C(O)OR, —OC(O)R (where R is hydrogen or is selected from an optionally substituted alkyl, alkenyl, alkynyl, cycloalkyl and aryl group), —C(O)NR′R″, —NR′C(O)R″ and —NR′R″ (where R′ and R″ are independently selected from hydrogen and lower alkyl);
or R⁸ and R⁹ are together ═O, ═S, ═NR or ═NOR, (where R is hydrogen or lower alkyl);
or R⁶ and R⁸ together form a bond;
or R⁴, R⁵, R⁶, R⁸ and R⁹ together with the atoms to which they are attached form an aromatic or heteroaromatic ring;
or R⁶, R⁷ and R⁸ and the atoms to which they are attached, together with a ring atom of B form a six membered aromatic or heteroaromatic ring fused to ring B;
m=0, 1 or 2;
each R¹⁰ is independently selected from hydrogen, cyano, halo, nitro, optionally substituted alkyl, optionally substituted alkenyl, optionally substituted alkynyl and optionally substituted cycloalkyl;
with the proviso that R³ is not —CH₂CO₂H when R¹ and R² are methoxy, m is 0, R⁴ and R⁵ together are ═O, R⁶ and R⁸ together form a bond, R⁷ an R⁹ are hydrogen, ring A is an unsubstituted furyl ring and B is an optional substituted phenyl ring;
and with the proviso that when R¹ and R² are methoxy, R³ is hydrogen, m is 0, R⁴ and R⁵ together are ═O, B is an optional substituted phenyl ring and one of R⁸ or R⁹ is hydrogen the other of R⁸ or R⁹ is not —CH₂CN or optionally substituted forms thereof;
and with the proviso that ring A is not an unsubstituted cyclopentadiene ring, when R¹ and R² are methoxy, R³ is hydrogen, R⁴ and R⁵ together are ═O, R⁶ and R⁸ together form a bond, R⁷ and R⁹ are hydrogen and B is an optionally substituted phenyl or pyridinyl ring;
and with the proviso that that R³ is not —(CH₂)₂NR′R″ (where R′ and R″ are independently hydrogen or alkyl, or together with the nitrogen to which they are attached form an unsubstituted piperidine ring), when R¹ and R² are methoxy, R⁴ is hydroxy, R⁵, R⁶, R⁷, R⁸ and R⁹ are hydrogen, ring A is a five membered heterocyclic ring containing oxygen, and B is an optionally substituted phenyl ring;
or a salt or pharmaceutically acceptable derivative thereof.

16. The compound of claim 15, or a salt or pharmaceutically acceptable derivative thereof, wherein the fused ring A is selected from: 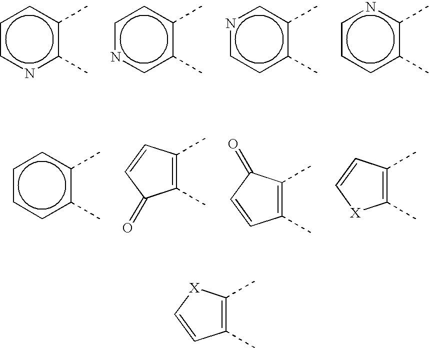
where X is O, S or NR and R is hydrogen, lower alkyl or oxygen; 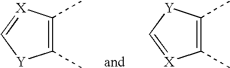
where X is N, and Y is O, S or NR and R is hydrogen, lower alkyl or oxygen;
and where the two dashed es on the right hand side of the rings indicate the location at which the ring is fused to the phenyl ring.

17. The compound of claim 16 or a salt or pharmaceutically acceptable derivative thereof wherein the fused ring A is an optionally substituted ring selected from the following:— 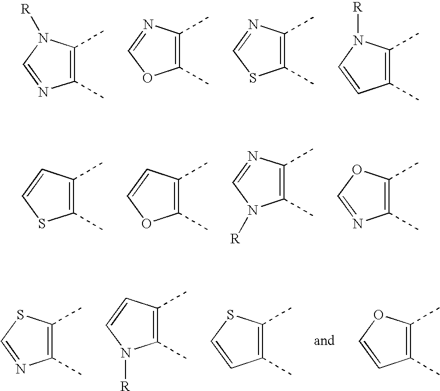
where R is hydrogen or lower alkyl.

18. A compound of formula I as defined in claim 16 or 17 wherein the fused ring A is optionally substituted with halo, lower alkyl, benzyl or —C(O)C₆H₅;
R¹ and R² are independently selected from hydrogen; halogen; hydroxy; lower alkoxy, optionally substituted benzyl, optionally substituted phenyl, optionally substituted diphenyl, optionally substituted phenoxy and optionally substituted benzoxy group;
R³ is hydrogen, methyl or benzyl optionally substituted with 1 to 3 halo or lower alkyl groups;
R⁴ d R⁵ are independently hydrogen or hydroxy, or together are ═O;
R⁶ is selected from hydrogen, halogen, —CN, —C(O)R (where R is lower alkyl or phenyl), —C(O)OR, (where R is hydrogen or lower alkyl), optionally substituted alkyl, and optionally substituted alkenyl group;
R⁷ is hydrogen;
R⁸ and R⁹ are independently selected from hydrogen; lower alkyl, —CHR(CN) (where R is selected from hydrogen, OH, lower alkyl and lower alkoxy), —C(O)R (where R is optionally substituted lower alkyl, optionally substituted lower alkoxy or optionally substituted phenyl), —NR′R″ (where R′ and R″ are independently selected from hydrogen or lower alkyl), and 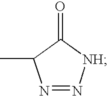
or R⁶ and R⁸ together form a bond between the carbons to which they are attached;
m is 0 or 1;
or a salt or pharmaceutically acceptable derivative thereof.

19. The compound of any one of claims 16 to 18 wherein B is an optionally substituted ring selected from phenyl, naphthalenyl, pyrrolyl, furyl, thiophenyl, imidazolyl, pyrazolyl, thiazolyl, oxazolyl, pyridinyl, pyryl, pyrimidinyl, indolyl, quinolinyl, isoquinolinyl and a ring system of the structure C 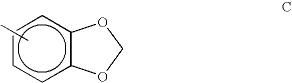
and the ring B is optionally substituted with one or more substituents independently selected from
h) halo, cyano, —NO₂, —SO₃, —OSO₃H, —OPO₃H₂, —PO₃ and —B(OH)₂;
i) —NR′R″ (where R′ and R″ are independently hydrogen or lower alkyl);
j) —NR′C(O)R″ (where R′ and R″ are independently hydrogen or lower alkyl);
k) phenyl and tetrazolyl;
l) —OR, —C(O)R, and —C(O)OR (where R is hydrogen, optionally substituted lower alkyl, optionally substituted phenyl, optionally substituted phenylloweralkyl and the optional substituents are independently selected from lower alkyl, halo and —NR′R″ where R′ and R″ are independently hydrogen or lower alkyl);
m) —C(O)NHSO₂R′″ and —S(O)₂NHC(O)R′″ (where R′″ lower alkyl);
n) optionally substituted lower alkyl such as —CH₃, —CH(CH₃)₂, —CH₂B(OH)₂, —CH₂PO₃, —CH₂SO₃, —CH₂OPO₃H₂, —CH₂OSO₃H, —CH₂C(O)NHSO₂R′″, —CH₂S(O)₂NHC(O)R′″ (where R′″ is lower alkyl), —CH₂C₆H₅, —CH₂-tetrazolyl, —(CH₂), NR′R″ (where n is from 1 to 4 and R′ and R″ are independently hydrogen or lower alkyl), —CF₃, —CF₂B(OH)₂, —CF₂PO₃, —CF₂SO₃, —CF₂OPO₃H₂, —CF₂OSO₃H, —CF₂C(O)NHSO₂R′″, —CF₂S(O)₂NHC(O)R′″ where R′″ is lower alkyl, —CF₂C₆H₅ and —CF₂-tetrazolyl.

20. The compound of claim 19 wherein ring B is substituted by —(CH₂)ₙR²⁰ where n is from 0 to 6 and R²⁰ is selected from —OSO₃H, —OPO₃H₂, —CO₂H, tetrazolyl, —B(OH)₂, —S(O)₂NHC(O)R′, —C(O)NHS(O)₂R′ (where R′ is lower alkyl), —OH, —C₆H₄OH, —CF₂PO₃ and —SO₃.

21. The compound of claim 19 or 20, or a salt or pharmaceutically acceptable derivative thereof, of the formula II 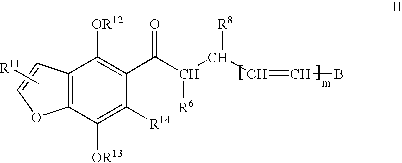
where R⁶ and R⁸ are hydrogen or together form a double bond, and R¹¹ is hydrogen, lower alkyl, halogen or —C(O)C₆H₅, R¹² and R¹³ are independently selected from hydrogen, alkyl, optionally substituted phenyl, optionally substituted benzyl, —(CH₂)ₙNR′R″ (where n is from 1 to 4 and R′ and R″ are independently hydrogen or lower alkyl) and —(CH₂)ₙR²⁰ (where m is from 1 to 4, and R²⁰ is selected from —OSO₃H, —OPO₃H₂, —CO₂H, tetrazolyl, —B(OH)₂, —S(O)₂NHC(O)R and —C(O)NHS(O)₂R (where R is lower alkyl)), R¹⁴ is hydroxy or alkoxy, m=0 or 1 and B is as defined in claim 19 or 20.

22. The compound of claim 21, or a salt or pharmaceutically acceptable derivative thereof, of the formula III 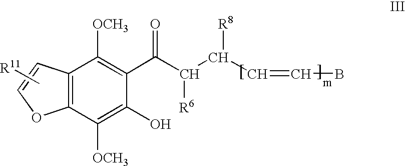
where R⁶, R⁸, R¹¹, m and B are as defined in claim 21.

23. The compound of claim 22, or a salt or pharmaceutically acceptable derivative thereof, of the formula IV 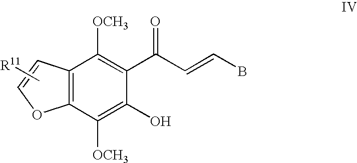
where R¹¹ is hydrogen, lower alkyl, halogen or —C(O)₆H₅ and B is an optionally substituted ring or ring system selected from phenyl, naphthalenyl pyridinyl, pyrrolyl, furyl, indolyl, quinolinyl, isoquinolinyl, thiophenyl,

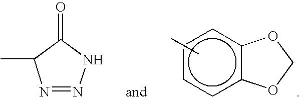

24. The compound of claim 23 herein B is optionally substituted with one or more substituents independently selected from —OPO₃H₂, —PO₃, —OSO₃, —SO₃, ⁻CH₂PO₃, —CH₂SO₃, —CO₂H, —CH₂CO₂H, —CF₂PO₃, —CF₂SO₃, —OH, —B(OH)₂, —OCH₃, —OCH₂CH₃, —CF₃, —CH₃, —CH₂CH₃, —CH(CH₃)₂, —C₆H₅, —OC₆H₅—OC₆H₄CH₃, -tetrazolyl, —CH₂tetrazolyl, —CF₂tetrazolyl, —NHC(O)CH₃, —F, —Cl, —Br, —CN, —OCH₂CH₂N(CH₂CH₃)₂,

25. The compound of claim 15, or a pharmaceutically acceptable derivative thereof, of the formula V 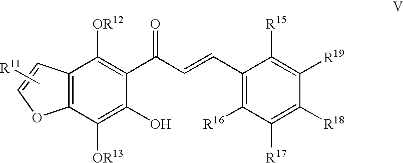
where R¹¹ is hydrogen, lower alkyl, halogen or —C(O)C₆H₅, R¹² and R¹³ are independently selected from hydrogen, alkyl, optionally substituted phenyl and optionally substituted benzyl; R¹³ also be selected from —(CH₂)ₙNR′R″ (where n is from 1 to 4 and R′ and R″ are independently hydrogen or lower alkyl) and —(CH₂)ₙR²⁰ (where n is from 0 to 6 and R²⁰ is selected from —OSO₃H, —OPO₃H₂, —CO₂H, -tetrazolyl, —B(OH)₂, —S(O)₂NHC(O)R and —C(O)NHS(O)₂R where R is lower alkyl);
R¹⁵, R¹⁶, R¹⁷ and R¹⁸ are independently selected from hydrogen, —OPO₃H₂, —PO₃, —OSO₃, —SO₃, —CH₂PO₃, —CH₂SO₃, —CO₂H, —CH₂CO₂H, —CF₂PO₃, —CF₂SO₃, —OH, —B(OH)₂, —OCH₃, —OCH₂CH₃, CF₃, —CH₃, —CH₃, —CH₂CH₃, —CH(CH₃)₂, —C₆H₅, —OC₆H₅—OC₆H₄CH₃, -tetrazolyl, —CH₂tetrazolyl, —CF₂tetrazolyl, —NHC(O)CH₃, —F, —Cl, —Br, —CN, —OCH₂CH₂N(CH₂CH₃)₂, —NO₂, —N(CH₃) and 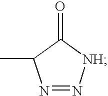
R¹⁹ is selected from —(CH₂)ₙR²⁰, where n is from 0 to 6, and R²⁰ is selected from hydrogen (when n is other than 0), —OSO₃H, —OPO₃H₂, —CO₂H, -tetrazolyl, —B(OH)₂, ⁻S(O)₂N(O)R′, —C(O)NHS(O)₂R′, —OR (where R′ is lower alkyl), —OR—C₆H₄OH, —CF₂PO₃ and —SO₃.

26. The compound of claim 15, or a pharmaceutically acceptable derivative thereof, of the formula VI 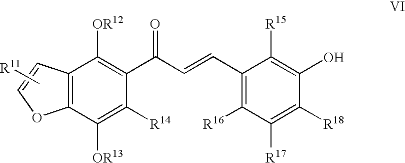
where R¹¹ is hydrogen, lower alkyl, halogen or —C(O)C₆H₅;
R¹² and R¹³ are independently selected from hydrogen, alkyl, optionally substituted phenyl and optionally substituted benzyl;
and R¹³ may also be selected from —(CH₂)ₙNR′R″ (where n is from 1 to 4 and R′ and R″ are independently hydrogen or lower alkyl) and —(CH₂)ₙR²⁰, (where n is from 0 to 6, and R²⁰ is selected from —OSO₃H, —OPO₃H₂, —CO₂H, tetrazolyl, —B(OH)₂, —S(O)₂NHC(O)R and —C(O)NHS(O)₂R, where R is lower alkyl);
R¹⁴ is hydroxy, alkoxy, —(CH)ₙNR′R″ (where n is from 1 to 4 and R′ and R″ are independently hydrogen or lower alkyl) or —(CH₂)ₙR²⁰, (where R²⁰ is selected from —OSO₃H, —OPO₃H₂, —CO₂H, tetrazolyl, —B(OH)₂, —S(O)₂NHC(O)R and —S(O)₂NHS(O)₂R where R is lower alkyl;
R¹⁵, R¹⁶, R¹⁷ and R¹⁸ are independently selected from hydrogen, —OPO₃H₂, —PO₃, —OSO₃, —SO₃, —CH₂P₃, —CH₂SO₃, —CO₂H, —CH₂CO₂H, —CF₂PO₃, —CF₂SO₃, —OH, —B(OH)₂, —OCH₃, —OCH₂CH₃, —CF₃, —CH₃, —CH₂CH₃, —CH(CH₃)₂, —C₆H₅, —OC₆H₅—OC₆H₄CH₃, -tetrazolyl, —CH₂tetrazolyl, —CF₂tetrazolyl, —NHC(O)CH₃, —F, —Cl, —Br, —CN, —OCH₂CH₂N(CH₂CH₃)₂, —NO₂, —N(CH₃) and

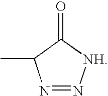

27. The compound of any one of claims 21 to 26 wherein R¹¹ is hydrogen.

28. The method of any one of claims 1 to 15 wherein the compound or its pharmaceutically acceptable derivative is administered to humans.

29. A pharmaceutical composition for use as an immunosuppressant, the composition comprising an effective amount of compound of any one of claims 15 to 27, or its pharmaceutically acceptable derivative thereof and optionally a carrier or diluent.

30. Use of a compound of formula I as defined in any one of claims 15 to 27 in the manufacture of a medicament for the treatment or prevention of autoimmune or chronic inflammatory diseases, or the prevention of rejection of foreign organ transplants and/or related afflictions.

31. Use as defined in claim 30 in the treatment or prevention of multiple sclerosis, rheumatoid arthritis or graft rejection.

32. Use of a compound as defined in any one of claims 15 to 27 for the treatment or prevention of a autoimmune or chronic inflammatory diseases, or the prevention of rejection of foreign organ transplants and/or related afflictions.

33. Use as define in claim 32 in the treatment or prevention of multiple sclerosis, rheumatoid arthritis or graft rejection.

34. A compound of formula I substantially as hereinbefore described with reference to the examples.

35. A process for the production of a compound of formula I as defined in claim 15, by reacting a compound of the formula VII with a compound of the formula VIII in the presence of sodium hydroxide, to produce a compound of the formula Ia, and optionally interconverting functional groups:— 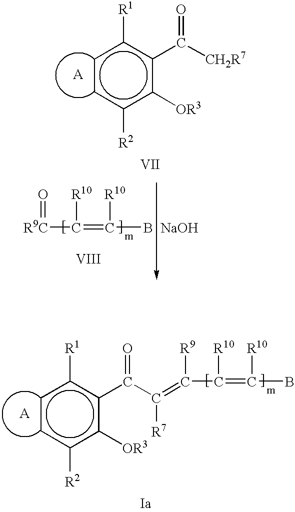
where ring A, R¹, R², R³, R⁷, R⁹, R¹⁰, B and m are as defined in claim 15.

36. A process for the production of a compound of formula I substantially as hereinbefore described with reference to the examples
